(19) United States
(12) Patent Application Publication
(10) Pub. No.: US 2023/0033976 A1
(43) Pub. Date: Feb. 2, 2023

(54) TECHNIQUES FOR IDENTIFYING RESTORATIVE MOMENTS

(71) Applicant: Oura Health Oy, Oulu (FI)

(72) Inventors: Hannu Kinnunen, Oulu (FI); Mari Karsikas, Oulu (FI); Heli Koskimäki, Oulu (FI); Mats Kyyrö, Oulu (FI)

(21) Appl. No.: 17/858,062

(22) Filed: Jul. 5, 2022

Related U.S. Application Data
(60) Provisional application No. 63/227,132, filed on Jul. 29, 2021.

(51) Int. Cl. G16H 40/63

(52) U.S. Cl. CPC G16H 40/63

(57) ABSTRACT

Methods, systems, and devices for restorative moment identification are described. A method may include receiving physiological data associated with a user from a wearable device, the physiological data including heart rate data and temperature data. The method may include identifying that the heart rate data is less than or equal to a heart rate threshold and identifying that the temperature data is within a temperature range of a baseline temperature associated with the user for at least a portion of a time interval. The method may include identifying a restorative moment for the time interval that the user is in a relaxed state based on the heart rate data being less than or equal to the heart rate threshold and the temperature data being within the temperature range of the baseline temperature, and causing a user device to display an indication of the restorative moment.

### CROSS REFERENCE

The present Application for Patent claims the benefit of U.S. Provisional Pat. Application No. 63/227,132 by KINNUNEN et al., entitled “TECHNIQUES FOR IDENTIFYING RESTORATIVE MOMENTS,” filed Jul. 29, 2021, assigned to the assignee hereof, and expressly incorporated by reference herein.

### FIELD OF TECHNOLOGY

The following relates to wearable devices and data processing, including techniques for identifying restorative moments.

### BACKGROUND

Some wearable devices may be configured to collect data from users associated with movement and other activities. For example, some wearable devices may be configured to detect when a user is at rest. However, conventional rest detection techniques implemented by some wearable devices are deficient.

### BRIEF DESCRIPTION OF THE DRAWINGS

FIG. 1 illustrates an example of a system that supports techniques for identifying restorative moments in accordance with aspects of the present disclosure.

FIG. 2 illustrates an example of a system that supports techniques for identifying restorative moments in accordance with aspects of the present disclosure.

FIG. 3 illustrates an example of a timing diagram that supports techniques for identifying restorative moments in accordance with aspects of the present disclosure.

FIG. 4 illustrates an example of a graphical user interface (GUI) that supports techniques for identifying restorative moments in accordance with aspects of the present disclosure.

FIG. 5 shows a block diagram of an apparatus that supports techniques for identifying restorative moments in accordance with aspects of the present disclosure.

FIG. 6 shows a block diagram of a wearable application that supports techniques for identifying restorative moments in accordance with aspects of the present disclosure.

FIG. 7 shows a diagram of a system including a device that supports techniques for identifying restorative moments in accordance with aspects of the present disclosure.

FIGS. 8 through 10 show flowcharts illustrating methods that support techniques for identifying restorative moments in accordance with aspects of the present disclosure.

### DETAILED DESCRIPTION

The human body is designed to heal and recover during periods of “rest,” such as when a user is sleeping. In particular, the human body may release hormones that help stimulate muscle repair and growth during periods of rest, such as during sleep. However, not all “rest” occurs during sleep. For example, a user may experience periods of rest throughout the day when they are awake, such as during periods of meditation or general relaxation. These periods of rest that are experienced while the user is awake may contribute to the user’s overall rest that is experienced throughout the day.

Some wearable devices may be configured to collect data from users associated with movement and other activities. For example, some wearable devices may be configured to detect when a user is active, when the user is sleeping, and when the user is resting. However, many wearable devices only monitor and detect periods of “rest” when a user is sleeping. Accordingly, such wearable devices may capture an incomplete picture of the user’s overall rest and recovery that is experienced throughout the day. In particular, such wearable devices may ignore or otherwise disregard periods of rest that the user experiences while they are awake. By only monitoring or detecting periods of rest while the user is asleep, such wearable devices may result in inaccurate or incomplete determinations as to the user’s restfulness (e.g., inaccurate Readiness Scores, inaccurate recovery scores). Incomplete or otherwise inaccurate Readiness Scores/recovery scores may result in a user over-estimating their recovery levels, which may lead to over-exertion, a de-prioritization of rest or sleep, or even injury.

Accordingly, techniques described herein are directed to wearable devices that are configured to identify periods of restfulness for a user while the user is awake. In other words, techniques described herein are directed to wearable devices that are configured to detect one or multiple “restorative durations” or “restorative moments” throughout the day. For the purposes of the present disclosure, the term “restorative moment,” “restorative time,” and like terms, may refer to a time duration when a user is inactive, or otherwise in a relaxed state. In this regard, the terms “restorative moment” and “restorative time” may refer to durations of time which provide a user with more energy and/or physical/mental readiness than is consumed for the

In order to efficiently and accurately track a user’s rest patterns, a wearable device may be configured to collect heart rate data and temperature data throughout a 24-hour period, including at night and during the daytime, and may be configured to identify “restorative moments” throughout the day based on the acquired physiological data. By identifying restorative moments for a user while the user is awake (in addition to tracking the user’s restfulness while the user is asleep), techniques described herein may enable a more complete and accurate determination of the user’s overall restfulness or recovery (e.g., more accurate Readiness Scores). Accordingly, techniques described herein may result in more accurate determination of Readiness Scores, which may enable wearable devices and systems to provide improved insights and guidance to the user which better correlate to the user’s overall recovery and how the user is actually feeling.

Aspects of the present disclosure are directed to techniques for detecting restorative moments based on data collected by a wearable device. In particular, aspects of the present disclosure are directed to techniques for identifying restorative moments, displaying an indication of the restorative moment, and selectively adjusting scores associated with the user (e.g., Readiness Scores) based on the restorative moments. For example, a system may receive data (e.g., temperature and heart rate), collected by a wearable device worn by a user, and may determine that the heart rate is less than or equal to a heart rate threshold for a time interval. In some cases, the system may determine that the temperature is within a temperature range of a baseline temperature associated with the user for the time interval. The restorative moment may be identified for the time interval that the user is in a relaxed state based on the heart rate being less than or equal to the heart rate threshold and the temperature being within the temperature range of the baseline temperature. Upon identifying the restorative moment, the system may cause the graphical user interface (GUI) of a user device coupled with the wearable device to display an indication of the restorative moment.

In some implementations, upon identifying the restorative moment, the system may prompt the user to confirm whether the user was in a relaxed state (e.g., whether the user was experiencing a restorative moment), and may selectively adjust the user’s future Readiness Scores when the user confirms that they were in a relaxed state. In some implementations, the system may generate messages (e.g., insights, alerts) for the user based on the identified restorative moment, where the alerts indicate how the identified restorative moment affected the user’s respective scores. The generated alerts may additionally, or alternatively, provide other insights associated with the restorative moment, such as whether the timing and/or duration of the restorative moment was beneficial for the user, whether the user should consider adjusting a timing and/or duration of restorative moments, and the like.

While much of the present disclosure is described in the context of updating Readiness Scores based on identified restorative moments, this is not to be regarded as a limitation of the present disclosure. Indeed, it is contemplated herein that data associated with a restorative moment of a user may be used to update any score, measure, metric, or other abstraction associated with a user’s health or activity. For example, historical restorative moment data for a user may be used to provide tailored guidance to the user regarding daily restorative moment targets for the user, guidance on how to improve restorative time, guidance on how to reduce stress or anxiety, and the like.

Aspects of the disclosure are initially described in the context of systems supporting physiological data collection from users via wearable devices. Additional aspects of the disclosure are described in the context of an example timing diagram and an example GUI. Aspects of the disclosure are further illustrated by and described with reference to apparatus diagrams, system diagrams, and flowcharts that relate to techniques for identifying restorative moments.

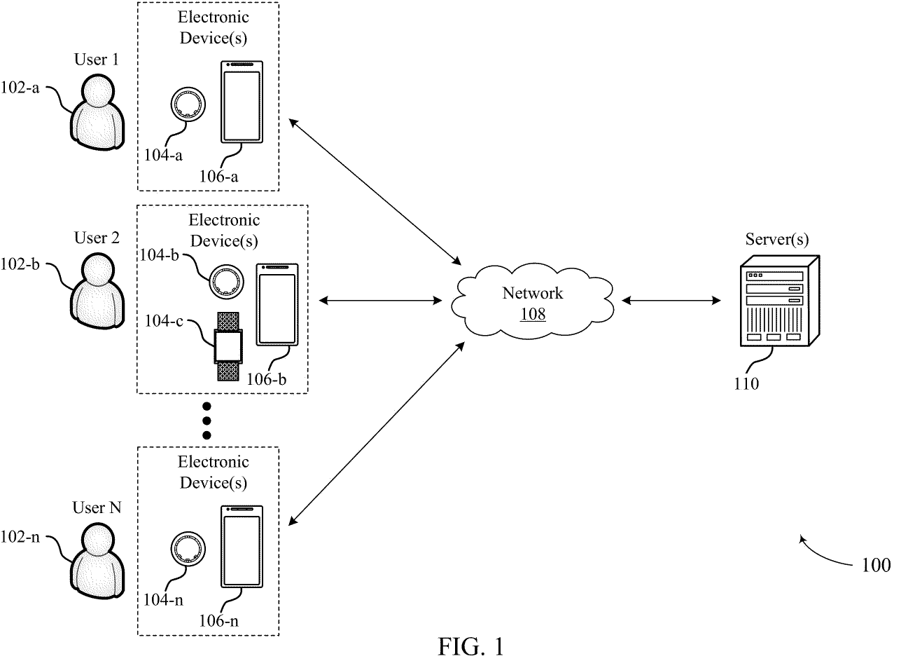

FIG. 1 illustrates an example of a system 100 that supports techniques for identifying restorative moments in accordance with aspects of the present disclosure. The system 100 includes a plurality of electronic devices (e.g., wearable devices 104, user devices 106) that may be worn and/or operated by one or more users 102. The system 100 further includes a network 108 and one or more servers 110.

The electronic devices may include any electronic devices known in the art, including wearable devices 104 (e.g., ring wearable devices, watch wearable devices, etc.), user devices 106 (e.g., smartphones, laptops, tablets). The electronic devices associated with the respective users 102 may include one or more of the following functionalities: 1) measuring physiological data, 2) storing the measured data, 3) processing the data, 4) providing outputs (e.g., via GUIs) to a user 102 based on the processed data, and 5) communicating data with one another and/or other computing devices. Different electronic devices may perform one or more of the functionalities.

Example wearable devices 104 may include wearable computing devices, such as a ring computing device (hereinafter “ring”) configured to be worn on a user’s 102 finger, a wrist computing device (e.g., a smart watch, fitness band, or bracelet) configured to be worn on a user’s 102 wrist, and/or a head mounted computing device (e.g., glasses/goggles). Wearable devices 104 may also include bands, straps (e.g., flexible or inflexible bands or straps), stick-on sensors, and the like, that may be positioned in other locations, such as bands around the head (e.g., a forehead headband), arm (e.g., a forearm band and/or bicep band), and/or leg (e.g., a thigh or calf band), behind the ear, under the armpit, and the like. Wearable devices 104 may also be attached to, or included in, articles of clothing. For example, wearable devices 104 may be included in pockets and/or pouches on clothing. As another example, wearable device 104 may be clipped and/or pinned to clothing. Example articles of clothing may include, but are not limited to, hats, shirts, gloves, pants, socks, outerwear (e.g., jackets), and undergarments. In some implementations, wearable devices 104 may be included with other types of devices such as training/sporting devices that are used during physical activity. For example, wearable devices 104 may be attached to, or included in, a bicycle, skis, a tennis racket, a golf club, and/or training weights.

Much of the present disclosure may be described in the context of a ring wearable device 104. Accordingly, the terms “ring 104,” “wearable device 104,” and like terms, may be used interchangeably, unless noted otherwise herein. However, the use of the term “ring 104” is not to be regarded as limiting, as it is contemplated herein that aspects of the present disclosure may be performed using other wearable devices (e.g., watch wearable devices, necklace wearable device, bracelet wearable devices, earring wearable devices, anklet wearable devices, and the like).

In some aspects, user devices 106 may include handheld mobile computing devices, such as smartphones and tablet computing devices. User devices 106 may also include personal computers, such as laptop and desktop computing devices. Other example user devices 106 may include server computing devices that may communicate with other electronic devices (e.g., via the Internet). In some implementations, computing devices may include medical devices, such as external wearable computing devices (e.g., Holter monitors). Medical devices may also include implantable medical devices, such as pacemakers and cardioverter defibrillators. Other example user devices 106 may include home computing devices, such as internet of things (IoT) devices (e.g., IoT devices), smart televisions, smart speakers, smart displays (e.g., video call displays), hubs (e.g., wireless communication hubs), security systems, smart appliances (e.g., thermostats and refrigerators), and fitness equipment.

Some electronic devices (e.g., wearable devices 104, user devices 106) may measure physiological parameters of respective users 102, such as photoplethysmography waveforms, continuous skin temperature, a pulse waveform, respiration rate, heart rate, heart rate variability (HRV), actigraphy, galvanic skin response, pulse oximetry, and/or other physiological parameters. Some electronic devices that measure physiological parameters may also perform some/all of the calculations described herein. Some electronic devices may not measure physiological parameters, but may perform some/all of the calculations described herein. For example, a ring (e.g., wearable device 104), user device application, or a server computing device may process received physiological data that was measured by other devices.

In some implementations, a user 102 may operate, or may be associated with, multiple electronic devices, some of which may measure physiological parameters and some of which may process the measured physiological parameters. In some implementations, a user 102 may have a ring (e.g., wearable device 104) that measures physiological parameters. The user 102 may also have, or be associated with, a user device 106 (e.g., mobile device, smartphone), where the wearable device 104 and the user device 106 are communicatively coupled to one another. In some cases, the user device 106 may receive data from the wearable device 104 and perform some/all of the calculations described herein. In some implementations, the user device 106 may also measure physiological parameters described herein, such as motion/activity parameters.

For example, as illustrated in FIG. 1, a first user 102-a (User 1) may operate, or may be associated with, a wearable device 104-a (e.g., ring 104-a) and a user device 106-a that may operate as described herein. In this example, the user device 106-a associated with user 102-a may process/store physiological parameters measured by the ring 104-a. Comparatively, a second user 102-b (User 2) may be associated with a ring 104-b, a watch wearable device 104-c (e.g., watch 104-c), and a user device 106-b, where the user device 106-b associated with user 102-b may process/store physiological parameters measured by the ring 104-b and/or the watch 104-c. Moreover, an nth user 102-n (User N) may be associated with an arrangement of electronic devices described herein (e.g., ring 104-n, user device 106-n). In some aspects, wearable devices 104 (e.g., rings 104, watches 104) and other electronic devices may be communicatively coupled to the user devices 106 of the respective users 102 via Bluetooth, Wi-Fi, and other wireless protocols.

The electronic devices of the system 100 (e.g., user devices 106, wearable devices 104) may be communicatively coupled to one or more servers 110 via wired or wireless communication protocols. For example, as shown in FIG. 1, the electronic devices (e.g., user devices 106) may be communicatively coupled to one or more servers 110 via a network 108. The network 108 may implement transfer control protocol and internet protocol (TCP/IP), such as the Internet, or may implement other network 108 protocols. Network connections between the network 108 and the respective electronic devices may facilitate transport of data via email, web, text messages, mail, or any other appropriate form of interaction within a computer network 108. For example, in some implementations, the ring 104-a associated with the first user 102-a may be communicatively coupled to the user device 106-a, where the user device 106-a is communicatively coupled to the servers 110 via the network 108. In additional or alternative cases, wearable devices 104 (e.g., rings 104, watches 104) may be directly communicatively coupled to the network 108.

The system 100 may offer an on-demand database service between the user devices 106 and the one or more servers 110. In some cases, the servers 110 may receive data from the user devices 106 via the network 108, and may store and analyze the data. Similarly, the servers 110 may provide data to the user devices 106 via the network 108. In some cases, the servers 110 may be located at one or more data centers. The servers 110 may be used for data storage, management, and processing. In some implementations, the servers 110 may provide a web-based interface to the user device 106 via web browsers.

In some aspects, the respective devices of the system 100 may support techniques for identifying restorative moments based on data collected by a wearable device. In particular, the system 100 illustrated in FIG. 1 may support techniques for identifying restorative moments for a user 102, and causing a user device 106 corresponding to the user 102 to display an indication of the identified restorative moment. For example, as shown in FIG. 1, User 1 (user 102-a) may be associated with a wearable device 104-a (e.g., ring 104-a) and a user device 106-a. In this example, the ring 104-a may collect data associated with the user 102-a, including temperature, heart rate, HRV, and the like. In some aspects, data collected by the ring 104-a may be used to determine whether User 1 has experienced (or is currently experiencing) a restorative moment. Identification of the restorative moment may be performed by any of the components of the system 100, including the ring 104-a, the user device 106-a associated with User 1, the one or more servers 110, or any combination thereof. Upon identifying the restorative moment, the system 100 may selectively cause the GUI of the user device 106-a to display an indication of the restorative moment.

In some implementations, upon identifying the restorative moment, the system 100 may prompt User 1 (e.g., via a GUI of the user device 106) to confirm whether the user 102-a was in a relaxed state or not, and may selectively adjust Readiness Scores for the user 102-a based on confirmation that the user was in a relaxed state. In some implementations, the system 100 may generate alerts for User 1 (e.g., via the ring 104-a, user device 106-a, or both) based on the identified restorative moment, where the alerts indicate how the restorative moment affected the respective scores. The generated alerts may additionally, or alternatively, provide other insights regarding the restorative moment, such as whether the timing and/or duration of the restorative moment was beneficial for User 1, whether User 1 should consider adjusting a timing and/or duration of the restorative moment, how the restorative moment affects stress/anxiety experienced by the user, and the like.

It should be appreciated by a person skilled in the art that one or more aspects of the disclosure may be implemented in a system 100 to additionally or alternatively solve other problems than those described above. Furthermore, aspects of the disclosure may provide technical improvements to “conventional” systems or processes as described herein. However, the description and appended drawings only include example technical improvements resulting from implementing aspects of the disclosure, and accordingly do not represent all of the technical improvements provided within the scope of the claims.

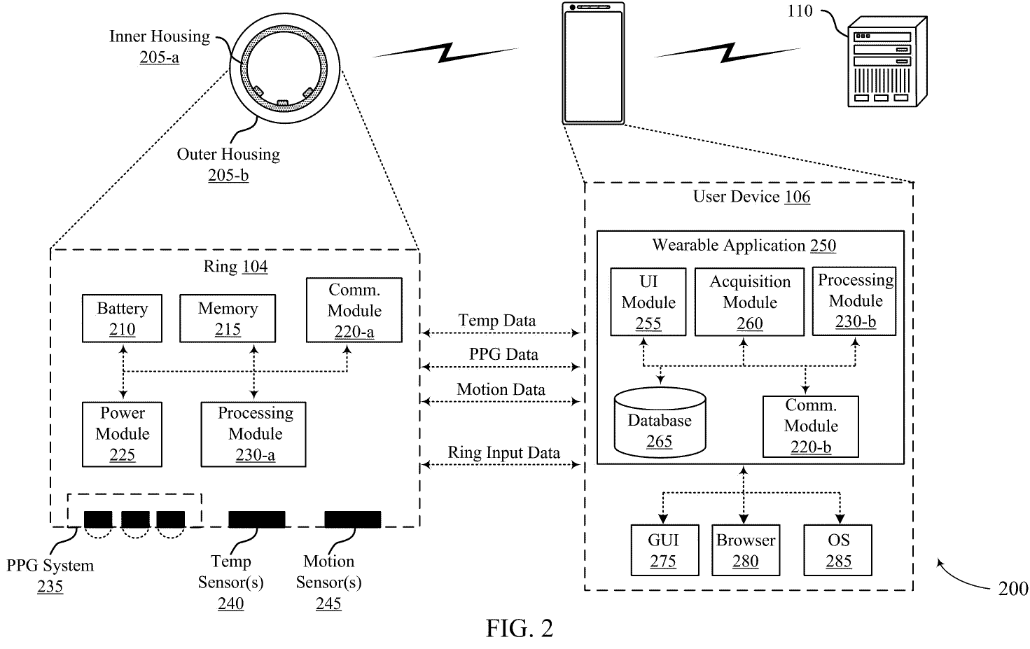

FIG. 2 illustrates an example of a system 200 that supports techniques for identifying restorative moments in accordance with aspects of the present disclosure. The system 200 may implement, or be implemented by, system 100. In particular, system 200 illustrates an example of a ring 104 (e.g., wearable device 104), a user device 106, and a server 110, as described with reference to FIG. 1.

In some aspects, the ring 104 may be configured to be worn around a user’s finger, and may determine one or more user physiological parameters when worn around the user’s finger. Example measurements and determinations may include, but are not limited to, user skin temperature, pulse waveforms, respiratory rate, heart rate, HRV, blood oxygen levels, and the like.

System 200 further includes a user device 106 (e.g., a smartphone) in communication with the ring 104. For example, the ring 104 may be in wireless and/or wired communication with the user device 106. In some implementations, the ring 104 may send measured and processed data (e.g., temperature data, photoplethysmogram (PPG) data, motion/accelerometer data, ring input data, and the like) to the user device 106. The user device 106 may also send data to the ring 104, such as ring 104 firmware/configuration updates. The user device 106 may process data. In some implementations, the user device 106 may transmit data to the server 110 for processing and/or storage.

The ring 104 may include a housing 205, which may include an inner housing 205-a and an outer housing 205-b. In some aspects, the housing 205 of the ring 104 may store or otherwise include various components of the ring including, but not limited to, device electronics, a power source (e.g., battery 210, and/or capacitor), one or more substrates (e.g., printable circuit boards) that interconnect the device electronics and/or power source, and the like. The device electronics may include device modules (e.g., hardware/software), such as: a processing module 230-a, a memory 215, a communication module 220-a, a power module 225, and the like. The device electronics may also include one or more sensors. Example sensors may include one or more temperature sensors 240, a PPG sensor assembly (e.g., PPG system 235), and one or more motion sensors 245.

The sensors may include associated modules (not illustrated) configured to communicate with the respective components/modules of the ring 104, and generate signals associated with the respective sensors. In some aspects, each of the components/modules of the ring 104 may be communicatively coupled to one another via wired or wireless connections. Moreover, the ring 104 may include additional and/or alternative sensors or other components that are configured to collect physiological data from the user, including light sensors (e.g., LEDs), oximeters, and the like.

The ring 104 shown and described with reference to FIG. 2 is provided solely for illustrative purposes. As such, the ring 104 may include additional or alternative components as those illustrated in FIG. 2. Other rings 104 that provide functionality described herein may be fabricated. For example, rings 104 with fewer components (e.g., sensors) may be fabricated. In a specific example, a ring 104 with a single temperature sensor 240 (or other sensor), a power source, and device electronics configured to read the single temperature sensor 240 (or other sensor) may be fabricated. In another specific example, a temperature sensor 240 (or other sensor) may be attached to a user’s finger (e.g., using a clamps, spring loaded clamps, etc.). In this case, the sensor may be wired to another computing device, such as a wrist worn computing device that reads the temperature sensor 240 (or other sensor). In other examples, a ring 104 that includes additional sensors and processing functionality may be fabricated.

The housing 205 may include one or more housing 205 components. The housing 205 may include an outer housing 205-b component (e.g., a shell) and an inner housing 205-a component (e.g., a molding). The housing 205 may include additional components (e.g., additional layers) not explicitly illustrated in FIG. 2. For example, in some implementations, the ring 104 may include one or more insulating layers that electrically insulate the device electronics and other conductive materials (e.g., electrical traces) from the outer housing 205-b (e.g., a metal outer housing 205-b). The housing 205 may provide structural support for the device electronics, battery 210, substrate(s), and other components. For example, the housing 205 may protect the device electronics, battery 210, and substrate(s) from mechanical forces, such as pressure and impacts. The housing 205 may also protect the device electronics, battery 210, and substrate(s) from water and/or other chemicals.

The outer housing 205-b may be fabricated from one or more materials. In some implementations, the outer housing 205-b may include a metal, such as titanium, that may provide strength and abrasion resistance at a relatively light weight. The outer housing 205-b may also be fabricated from other materials, such polymers. In some implementations, the outer housing 205-b may be protective as well as decorative.

The inner housing 205-a may be configured to interface with the user’s finger. The inner housing 205-a may be formed from a polymer (e.g., a medical grade polymer) or other material. In some implementations, the inner housing 205-a may be transparent. For example, the inner housing 205-a may be transparent to light emitted by the PPG light emitting diodes (LEDs). In some implementations, the inner housing 205-a component may be molded onto the outer housing 205-a. For example, the inner housing 205-a may include a polymer that is molded (e.g., injection molded) to fit into an outer housing 205-b metallic shell.

The ring 104 may include one or more substrates (not illustrated). The device electronics and battery 210 may be included on the one or more substrates. For example, the device electronics and battery 210 may be mounted on one or more substrates. Example substrates may include one or more printed circuit boards (PCBs), such as flexible PCB (e.g., polyimide). In some implementations, the electronics/battery 210 may include surface mounted devices (e.g., surface-mount technology (SMT) devices) on a flexible PCB. In some implementations, the one or more substrates (e.g., one or more flexible PCBs) may include electrical traces that provide electrical communication between device electronics. The electrical traces may also connect the battery 210 to the device electronics.

The device electronics, battery 210, and substrates may be arranged in the ring 104 in a variety of ways. In some implementations, one substrate that includes device electronics may be mounted along the bottom of the ring 104 (e.g., the bottom half), such that the sensors (e.g., PPG system 235, temperature sensors 240, motion sensors 245, and other sensors) interface with the underside of the user’s finger. In these implementations, the battery 210 may be included along the top portion of the ring 104 (e.g., on another substrate).

The various components/modules of the ring 104 represent functionality (e.g., circuits and other components) that may be included in the ring 104. Modules may include any discrete and/or integrated electronic circuit components that implement analog and/or digital circuits capable of producing the functions attributed to the modules herein. For example, the modules may include analog circuits (e.g., amplification circuits, filtering circuits, analog/digital conversion circuits, and/or other signal conditioning circuits). The modules may also include digital circuits (e.g., combinational or sequential logic circuits, memory circuits etc.).

The memory 215 (memory module) of the ring 104 may include any volatile, non-volatile, magnetic, or electrical media, such as a random access memory (RAM), read-only memory (ROM), non-volatile RAM (NVRAM), electrically-erasable programmable ROM (EEPROM), flash memory, or any other memory device. The memory 215 may store any of the data described herein. For example, the memory 215 may be configured to store data (e.g., motion data, temperature data, PPG data) collected by the respective sensors and PPG system 235. Furthermore, memory 215 may include instructions that, when executed by one or more processing circuits, cause the modules to perform various functions attributed to the modules herein. The device electronics of the ring 104 described herein are only example device electronics. As such, the types of electronic components used to implement the device electronics may vary based on design considerations.

The functions attributed to the modules of the ring 104 described herein may be embodied as one or more processors, hardware, firmware, software, or any combination thereof. Depiction of different features as modules is intended to highlight different functional aspects and does not necessarily imply that such modules must be realized by separate hardware/software components. Rather, functionality associated with one or more modules may be performed by separate hardware/software components or integrated within common hardware/software components.

The processing module 230-a of the ring 104 may include one or more processors (e.g., processing units), microcontrollers, digital signal processors, systems on a chip (SOCs), and/or other processing devices. The processing module 230-a communicates with the modules included in the ring 104. For example, the processing module 230-a may transmit/receive data to/from the modules and other components of the ring 104, such as the sensors. As described herein, the modules may be implemented by various circuit components. Accordingly, the modules may also be referred to as circuits (e.g., a communication circuit and power circuit).

The processing module 230-a may communicate with the memory 215. The memory 215 may include computer-readable instructions that, when executed by the processing module 230-a, cause the processing module 230-a to perform the various functions attributed to the processing module 230-a herein. In some implementations, the processing module 230-a (e.g., a microcontroller) may include additional features associated with other modules, such as communication functionality provided by the communication module 220-a (e.g., an integrated Bluetooth Low Energy transceiver) and/or additional onboard memory 215.

The communication module 220-a may include circuits that provide wireless and/or wired communication with the user device 106 (e.g., communication module 220-b of the user device 106). In some implementations, the communication modules 220-a, 220-b may include wireless communication circuits, such as Bluetooth circuits and/or Wi-Fi circuits. In some implementations, the communication modules 220-a, 220-b can include wired communication circuits, such as Universal Serial Bus (USB) communication circuits. Using the communication module 220-a, the ring 104 and the user device 106 may be configured to communicate with each other. The processing module 230-a of the ring may be configured to transmit/receive data to/from the user device 106 via the communication module 220-a. Example data may include, but is not limited to, motion data, temperature data, pulse waveforms, heart rate data, HRV data, PPG data, and status updates (e.g., charging status, battery charge level, and/or ring 104 configuration settings). The processing module 230-a of the ring may also be configured to receive updates (e.g., software/firmware updates) and data from the user device 106.

The ring 104 may include a battery 210 (e.g., a rechargeable battery 210). An example battery 210 may include a Lithium-Ion or Lithium-Polymer type battery 210, although a variety of battery 210 options are possible. The battery 210 may be wirelessly charged. In some implementations, the ring 104 may include a power source other than the battery 210, such as a capacitor. The power source (e.g., battery 210 or capacitor) may have a curved geometry that matches the curve of the ring 104. In some aspects, a charger or other power source may include additional sensors that may be used to collect data in addition to, or which supplements, data collected by the ring 104 itself. Moreover, a charger or other power source for the ring 104 may function as a user device 106, in which case the charger or other power source for the ring 104 may be configured to receive data from the ring 104, store and/or process data received from the ring 104, and communicate data between the ring 104 and the servers 110.

In some aspects, the ring 104 includes a power module 225 that may control charging of the battery 210. For example, the power module 225 may interface with an external wireless charger that charges the battery 210 when interfaced with the ring 104. The charger may include a datum structure that mates with a ring 104 datum structure to create a specified orientation with the ring 104 during 104 charging. The power module 225 may also regulate voltage(s) of the device electronics, regulate power output to the device electronics, and monitor the state of charge of the battery 210. In some implementations, the battery 210 may include a protection circuit module (PCM) that protects the battery 210 from high current discharge, over voltage during 104 charging, and under voltage during 104 discharge. The power module 225 may also include electro-static discharge (ESD) protection.

The one or more temperature sensors 240 may be electrically coupled to the processing module 230-a. The temperature sensor 240 may be configured to generate a temperature signal (e.g., temperature data) that indicates a temperature read or sensed by the temperature sensor 240. The processing module 230-a may determine a temperature of the user in the location of the temperature sensor 240. For example, in the ring 104, temperature data generated by the temperature sensor 240 may indicate a temperature of a user at the user’s finger (e.g., skin temperature). In some implementations, the temperature sensor 240 may contact the user’s skin. In other implementations, a portion of the housing 205 (e.g., the inner housing 205-a) may form a barrier (e.g., a thin, thermally conductive barrier) between the temperature sensor 240 and the user’s skin. In some implementations, portions of the ring 104 configured to contact the user’s finger may have thermally conductive portions and thermally insulative portions. The thermally conductive portions may conduct heat from the user’s finger to the temperature sensors 240. The thermally insulative portions may insulate portions of the ring 104 (e.g., the temperature sensor 240) from ambient temperature.

In some implementations, the temperature sensor 240 may generate a digital signal (e.g., temperature data) that the processing module 230-a may use to determine the temperature. As another example, in cases where the temperature sensor 240 includes a passive sensor, the processing module 230-a (or a temperature sensor 240 module) may measure a current/voltage generated by the temperature sensor 240 and determine the temperature based on the measured current/voltage. Example temperature sensors 240 may include a thermistor, such as a negative temperature coefficient (NTC) thermistor, or other types of sensors including resistors, transistors, diodes, and/or other electrical/electronic components.

The processing module 230-a may sample the user’s temperature over time. For example, the processing module 230-a may sample the user’s temperature according to a sampling rate. An example sampling rate may include one sample per second, although the processing module 230-a may be configured to sample the temperature signal at other sampling rates that are higher or lower than one sample per second. In some implementations, the processing module 230-a may sample the user’s temperature continuously throughout the day and night. Sampling at a sufficient rate (e.g., one sample per second) throughout the day may provide sufficient temperature data for analysis described herein.

The processing module 230-a may store the sampled temperature data in memory 215. In some implementations, the processing module 230-a may process the sampled temperature data. For example, the processing module 230-a may determine average temperature values over a period of time. In one example, the processing module 230-a may determine an average temperature value each minute by summing all temperature values collected over the minute and dividing by the number of samples over the minute. In a specific example where the temperature is sampled at one sample per second, the average temperature may be a sum of all sampled temperatures for one minute divided by sixty seconds. The memory 215 may store the average temperature values over time. In some implementations, the memory 215 may store average temperatures (e.g., one per minute) instead of sampled temperatures in order to conserve memory 215.

The sampling rate, which may be stored in memory 215, may be configurable. In some implementations, the sampling rate may be the same throughout the day and night. In other implementations, the sampling rate may be changed throughout the day/night. In some implementations, the ring 104 may filter/reject temperature readings, such as large spikes in temperature that are not indicative of physiological changes (e.g., a temperature spike from a hot shower). In some implementations, the ring 104 may filter/reject temperature readings that may not be reliable due to other factors, such as excessive motion during 104 exercise (e.g., as indicated by a motion sensor 245).

The ring 104 (e.g., communication module) may transmit the sampled and/or average temperature data to the user device 106 for storage and/or further processing. The user device 106 may transfer the sampled and/or average temperature data to the server 110 for storage and/or further processing.

Although the ring 104 is illustrated as including a single temperature sensor 240, the ring 104 may include multiple temperature sensors 240 in one or more locations, such as arranged along the inner housing 205-a near the user’s finger. In some implementations, the temperature sensors 240 may be stand-alone temperature sensors 240. Additionally, or alternatively, one or more temperature sensors 240 may be included with other components (e.g., packaged with other components), such as with the accelerometer and/or processor.

The processing module 230-a may acquire and process data from multiple temperature sensors 240 in a similar manner described with respect to a single temperature sensor 240. For example, the processing module 230 may individually sample, average, and store temperature data from each of the multiple temperature sensors 240. In other examples, the processing module 230-a may sample the sensors at different rates and average/store different values for the different sensors. In some implementations, the processing module 230-a may be configured to determine a single temperature based on the average of two or more temperatures determined by two or more temperature sensors 240 in different locations on the finger.

The temperature sensors 240 on the ring 104 may acquire distal temperatures at the user’s finger (e.g., any finger). For example, one or more temperature sensors 240 on the ring 104 may acquire a user’s temperature from the underside of a finger or at a different location on the finger. In some implementations, the ring 104 may continuously acquire distal temperature (e.g., at a sampling rate). Although distal temperature measured by a ring 104 at the finger is described herein, other devices may measure temperature at the same/different locations. In some cases, the distal temperature measured at a user’s finger may differ from the temperature measured at a user’s wrist or other external body location. Additionally, the distal temperature measured at a user’s finger (e.g., a “shell” temperature) may differ from the user’s core temperature. As such, the ring 104 may provide a useful temperature signal that may not be acquired at other internal/external locations of the body. In some cases, continuous temperature measurement at the finger may capture temperature fluctuations (e.g., small or large fluctuations) that may not be evident in core temperature. For example, continuous temperature measurement at the finger may capture minute-to-minute or hour-to-hour temperature fluctuations that provide additional insight that may not be provided by other temperature measurements elsewhere in the body.

The ring 104 may include a PPG system 235. The PPG system 235 may include one or more optical transmitters that transmit light. The PPG system 235 may also include one or more optical receivers that receive light transmitted by the one or more optical transmitters. An optical receiver may generate a signal (hereinafter “PPG” signal) that indicates an amount of light received by the optical receiver. The optical transmitters may illuminate a region of the user’s finger. The PPG signal generated by the PPG system 235 may indicate the perfusion of blood in the illuminated region. For example, the PPG signal may indicate blood volume changes in the illuminated region caused by a user’s pulse pressure. The processing module 230-a may sample the PPG signal and determine a user’s pulse waveform based on the PPG signal. The processing module 230-a may determine a variety of physiological parameters based on the user’s pulse waveform, such as a user’s respiratory rate, heart rate, HRV, oxygen saturation, and other circulatory parameters.

In some implementations, the PPG system 235 may be configured as a reflective PPG system 235 in which the optical receiver(s) receive transmitted light that is reflected through the region of the user’s finger. In some implementations, the PPG system 235 may be configured as a transmissive PPG system 235 in which the optical transmitter(s) and optical receiver(s) are arranged opposite to one another, such that light is transmitted directly through a portion of the user’s finger to the optical receiver(s).

The number and ratio of transmitters and receivers included in the PPG system 235 may vary. Example optical transmitters may include light-emitting diodes (LEDs). The optical transmitters may transmit light in the infrared spectrum and/or other spectrums. Example optical receivers may include, but are not limited to, photosensors, phototransistors, and photodiodes. The optical receivers may be configured to generate PPG signals in response to the wavelengths received from the optical transmitters. The location of the transmitters and receivers may vary. Additionally, a single device may include reflective and/or transmissive PPG systems 235.

The PPG system 235 illustrated in FIG. 2 may include a reflective PPG system 235 in some implementations. In these implementations, the PPG system 235 may include a centrally located optical receiver (e.g., at the bottom of the ring 104) and two optical transmitters located on each side of the optical receiver. In this implementation, the PPG system 235 (e.g., optical receiver) may generate the PPG signal based on light received from one or both of the optical transmitters. In other implementations, other placements, combinations, and/or configurations of one or more optical transmitters and/or optical receivers are contemplated.

The processing module 230-a may control one or both of the optical transmitters to transmit light while sampling the PPG signal generated by the optical receiver. In some implementations, the processing module 230-a may cause the optical transmitter with the stronger received signal to transmit light while sampling the PPG signal generated by the optical receiver. For example, the selected optical transmitter may continuously emit light while the PPG signal is sampled at a sampling rate (e.g., 250 Hz).

Sampling the PPG signal generated by the PPG system 235 may result in a pulse waveform, which may be referred to as a “PPG.” The pulse waveform may indicate blood pressure vs time for multiple cardiac cycles. The pulse waveform may include peaks that indicate cardiac cycles. Additionally, the pulse waveform may include respiratory induced variations that may be used to determine respiration rate. The processing module 230-a may store the pulse waveform in memory 215 in some implementations. The processing module 230-a may process the pulse waveform as it is generated and/or from memory 215 to determine user physiological parameters described herein.

The processing module 230-a may determine the user’s heart rate based on the pulse waveform. For example, the processing module 230-a may determine heart rate (e.g., in beats per minute) based on the time between peaks in the pulse waveform. The time between peaks may be referred to as an interbeat interval (IBI). The processing module 230-a may store the determined heart rate values and IBI values in memory 215.

The processing module 230-a may determine HRV over time. For example, the processing module 230-a may determine HRV based on the variation in the IBls. The processing module 230-a may store the HRV values over time in the memory 215. Moreover, the processing module 230-a may determine the user’s respiratory rate over time. For example, the processing module 230-a may determine respiratory rate based on frequency modulation, amplitude modulation, or baseline modulation of the user’s IBI values over a period of time. Respiratory rate may be calculated in breaths per minute or as another breathing rate (e.g., breaths per 30 seconds). The processing module 230-a may store user respiratory rate values over time in the memory 215.

The ring 104 may include one or more motion sensors 245, such as one or more accelerometers (e.g., 6-D accelerometers) and/or one or more gyroscopes (gyros). The motion sensors 245 may generate motion signals that indicate motion of the sensors. For example, the ring 104 may include one or more accelerometers that generate acceleration signals that indicate acceleration of the accelerometers. As another example, the ring 104 may include one or more gyro sensors that generate gyro signals that indicate angular motion (e.g., angular velocity) and/or changes in orientation. The motion sensors 245 may be included in one or more sensor packages. An example accelerometer/gyro sensor is a Bosch BMl160 inertial micro electro-mechanical system (MEMS) sensor that may measure angular rates and accelerations in three perpendicular axes.

The processing module 230-a may sample the motion signals at a sampling rate (e.g., 50 Hz) and determine the motion of the ring 104 based on the sampled motion signals. For example, the processing module 230-a may sample acceleration signals to determine acceleration of the ring 104. As another example, the processing module 230-a may sample a gyro signal to determine angular motion. In some implementations, the processing module 230-a may store motion data in memory 215. Motion data may include sampled motion data as well as motion data that is calculated based on the sampled motion signals (e.g., acceleration and angular values).

The ring 104 may store a variety of data described herein. For example, the ring 104 may store temperature data, such as raw sampled temperature data and calculated temperature data (e.g., average temperatures). As another example, the ring 104 may store PPG signal data, such as pulse waveforms and data calculated based on the pulse waveforms (e.g., heart rate values, IBI values, HRV values, and respiratory rate values). The ring 104 may also store motion data, such as sampled motion data that indicates linear and angular motion.

The ring 104, or other computing device, may calculate and store additional values based on the sampled/calculated physiological data. For example, the processing module 230 may calculate and store various metrics, such as sleep metrics (e.g., a Sleep Score), activity metrics, and readiness metrics. In some implementations, additional values/metrics may be referred to as “derived values.” The ring 104, or other computing/wearable device, may calculate a variety of values/metrics with respect to motion. Example derived values for motion data may include, but are not limited to, motion count values, regularity values, intensity values, metabolic equivalence of task values (METs), and orientation values. Motion counts, regularity values, intensity values, and METs may indicate an amount of user motion (e.g., velocity/acceleration) over time. Orientation values may indicate how the ring 104 is oriented on the user’s finger and if the ring 104 is worn on the left hand or right hand.

In some implementations, motion counts and regularity values may be determined by counting a number of acceleration peaks within one or more periods of time (e.g., one or more 30 second to 1 minute periods). Intensity values may indicate a number of movements and the associated intensity (e.g., acceleration values) of the movements. The intensity values may be categorized as low, medium, and high, depending on associated threshold acceleration values. METs may be determined based on the intensity of movements during a period of time (e.g., 30 seconds), the regularity/irregularity of the movements, and the number of movements associated with the different intensities.

In some implementations, the processing module 230-a may compress the data stored in memory 215. For example, the processing module 230-a may delete sampled data after making calculations based on the sampled data. As another example, the processing module 230-a may average data over longer periods of time in order to reduce the number of stored values. In a specific example, if average temperatures for a user over one minute are stored in memory 215, the processing module 230-a may calculate average temperatures over a five minute time period for storage, and then subsequently erase the one minute average temperature data. The processing module 230-a may compress data based on a variety of factors, such as the total amount of used/available memory 215 and/or an elapsed time since the ring 104 last transmitted the data to the user device 106.

Although a user’s physiological parameters may be measured by sensors included on a ring 104, other devices may measure a user’s physiological parameters. For example, although a user’s temperature may be measured by a temperature sensor 240 included in a ring 104, other devices may measure a user’s temperature. In some examples, other wearable devices (e.g., wrist devices) may include sensors that measure user physiological parameters. Additionally, medical devices, such as external medical devices (e.g., wearable medical devices) and/or implantable medical devices, may measure a user’s physiological parameters. One or more sensors on any type of computing device may be used to implement the techniques described herein.

The physiological measurements may be taken continuously throughout the day and/or night. In some implementations, the physiological measurements may be taken during 104 portions of the day and/or portions of the night. In some implementations, the physiological measurements may be taken in response to determining that the user is in a specific state, such as an active state, resting state, and/or a sleeping state. For example, the ring 104 can make physiological measurements in a resting/sleep state in order to acquire cleaner physiological signals. In one example, the ring 104 or other device/system may detect when a user is resting and/or sleeping and acquire physiological parameters (e.g., temperature) for that detected state. The devices/systems may use the resting/sleep physiological data and/or other data when the user is in other states in order to implement the techniques of the present disclosure.

In some implementations, as described previously herein, the ring 104 may be configured to collect, store, and/or process data, and may transfer any of the data described herein to the user device 106 for storage and/or processing. In some aspects, the user device 106 includes a wearable application 250, an operating system (OS), a web browser application (e.g., web browser 280), one or more additional applications, and a GUI 275. The user device 106 may further include other modules and components, including sensors, audio devices, haptic feedback devices, and the like. The wearable application 250 may include an example of an application (e.g., “app”) that may be installed on the user device 106. The wearable application 250 may be configured to acquire data from the ring 104, store the acquired data, and process the acquired data as described herein. For example, the wearable application 250 may include a user interface (UI) module 255, an acquisition module 260, a processing module 230-b, a communication module 220-b, and a storage module (e.g., database 265) configured to store application data.

The various data processing operations described herein may be performed by the ring 104, the user device 106, the servers 110, or any combination thereof. For example, in some cases, data collected by the ring 104 may be pre-processed and transmitted to the user device 106. In this example, the user device 106 may perform some data processing operations on the received data, may transmit the data to the servers 110 for data processing, or both. For instance, in some cases, the user device 106 may perform processing operations that require relatively low processing power and/or operations that require a relatively low latency, whereas the user device 106 may transmit the data to the servers 110 for processing operations that require relatively high processing power and/or operations that may allow relatively higher latency.

In some aspects, the ring 104, user device 106, and server 110 of the system 200 may be configured to evaluate sleep patterns for a user. In particular, the respective components of the system 200 may be used to collect data from a user via the ring 104, and generate one or more scores (e.g., Sleep Score, Readiness Score) for the user based on the collected data. For example, as noted previously herein, the ring 104 of the system 200 may be worn by a user to collect data from the user, including temperature, heart rate, HRV, and the like. Data collected by the ring 104 may be used to determine when the user is asleep in order to evaluate the user’s sleep for a given “sleep day.” In some aspects, scores may be calculated for the user for each respective sleep day, such that a first sleep day is associated with a first set of scores, and a second sleep day is associated with a second set of scores. Scores may be calculated for each respective sleep day based on data collected by the ring 104 during the respective sleep day. Scores may include, but are not limited to, Sleep Scores, Readiness Scores, and the like.

In some cases, “sleep days” may align with the traditional calendar days, such that a given sleep day runs from midnight to midnight of the respective calendar day. In other cases, sleep days may be offset relative to calendar days. For example, sleep days may run from 6:00 pm (18:00) of a calendar day until 6:00 pm (18:00) of the subsequent calendar day. In this example, 6:00 pm may serve as a “cut-off time,” where data collected from the user before 6:00 pm is counted for the current sleep day, and data collected from the user after 6:00 pm is counted for the subsequent sleep day. Due to the fact that most individuals sleep the most at night, offsetting sleep days relative to calendar days may enable the system 200 to evaluate sleep patterns for users in such a manner that is consistent with their sleep schedules. In some cases, users may be able to selectively adjust (e.g., via the GUI) a timing of sleep days relative to calendar days so that the sleep days are aligned with the duration of time that the respective users typically sleep.

In some implementations, each overall score for a user for each respective day (e.g., Sleep Score, Readiness Score) may be determined/calculated based on one or more “contributors,” “factors,” or “contributing factors.” For example, a user’s overall Sleep Score may be calculated based on a set of contributors, including: total sleep, efficiency, restfulness, rapid eye movement (REM) sleep, deep sleep, latency, timing, or any combination thereof. The Sleep Score may include any quantity of contributors. The “total sleep” contributor may refer to the sum of all sleep periods of the sleep day. The “efficiency” contributor may reflect the percentage of time spent asleep compared to time spent awake while in bed, and may be calculated using the efficiency average of long sleep periods (e.g., primary sleep period) of the sleep day, weighted by a duration of each sleep period. The “restfulness” contributor may indicate how restful the user’s sleep is, and may be calculated using the average of all sleep periods of the sleep day, weighted by a duration of each period. The restfulness contributor may be based on a “wake up count” (e.g., sum of all the wake-ups (when user wakes up) detected during different sleep periods), excessive movement, and a “got up count” (e.g., sum of all the got-ups (when user gets out of bed) detected during the different sleep periods).

The “REM sleep” contributor may refer to a sum total of REM sleep durations across all sleep periods of the sleep day including REM sleep. Similarly, the “deep sleep” contributor may refer to a sum total of deep sleep durations across all sleep periods of the sleep day including deep sleep. The “latency” contributor may signify how long (e.g., average, median, longest) the user takes to go to sleep, and may be calculated using the average of long sleep periods throughout the sleep day, weighted by a duration of each period. Lastly, the “timing” contributor may refer to a relative timing of sleep periods within the sleep day and/or calendar day, and may be calculated using the average of all sleep periods of the sleep day, weighted by a duration of each period.

By way of another example, a user’s overall Readiness Score may be calculated based on a set of contributors, including: sleep, sleep balance, heart rate, HRV balance, recovery index, temperature, activity, activity balance, or any combination thereof. The Readiness Score may include any quantity of contributors. The “sleep” contributor may refer to the combined Sleep Score of all sleep periods within the sleep day. The “sleep balance” contributor may refer to a cumulative duration of all sleep periods within the sleep day. In particular, sleep balance may indicate to a user whether the sleep that the user has been getting over some duration of time (e.g., the past two weeks) is in balance with the user’s needs. Typically, adults need 7-9 hours of sleep a night to stay healthy, alert, and to perform at their best both mentally and physically. However, it is normal to have an occasional night of bad sleep, so the sleep balance contributor takes into account long-term sleep patterns to determine whether each user’s sleep needs are being met. The “resting heart rate” contributor may indicate a lowest heart rate from the longest sleep period of the sleep day (e.g., primary sleep period) and/or the lowest heart rate from naps occurring after the primary sleep period.

Continuing with reference to the “contributors” (e.g., factors, contributing factors) of the Readiness Score, the “HRV balance” contributor may indicate a highest HRV average from the primary sleep period and the naps happening after the primary sleep period. The HRV balance contributor may help users keep track of their recovery status by comparing their HRV trend over a first time period (e.g., two weeks) to an average HRV over some second, longer time period (e.g., three months). The “recovery index” contributor may be calculated based on the longest sleep period. Recovery index measures how long it takes for a user’s resting heart rate to stabilize during the night. A sign of a very good recovery is that the user’s resting heart rate stabilizes during the first half of the night, at least six hours before the user wakes up, leaving the body time to recover for the next day. The “body temperature” contributor may be calculated based on the longest sleep period (e.g., primary sleep period) or based on a nap happening after the longest sleep period if the user’s highest temperature during the nap is at least 0.5° C. higher than the highest temperature during the longest period. In some aspects, the ring may measure a user’s body temperature while the user is asleep, and the system 200 may display the user’s average temperature relative to the user’s baseline temperature. If a user’s body temperature is outside of their normal range (e.g., clearly above or below 0.0), the body temperature contributor may be highlighted (e.g., go to a “Pay attention” state) or otherwise generate an alert for the user.

In some aspects, the ring 104, user device 106, and servers 110 of the system 200 may be configured to identify restorative moments for a user. In particular, the respective components of the system 200 may be used to determine what effect restorative moments have on respective scores (e.g., Readiness Scores) for a user, stress/anxiety experienced by the user, and the like. The moments the user is in a relaxed state may be detected by leveraging temperature sensors on the ring of the system 200. In some cases, the restorative moments may be indicated by a quantity of minutes of relaxation experienced by the user that may be identified on an individual basis rather than compared to other users.

For example, as noted previously herein, the ring 104 of the system 200 may be worn by a user to collect data from the user, including temperature, heart rate, movement, and the like. The ring of the system 200 may collect the physiological data from the user based on arterial blood flow. Data collected by the ring 104 may be used to determine when the user is in a relaxed state and estimate daytime restorative moments for the daytime so that the moments when the user is in a relaxed state may be detected by the system 200. While much of the present disclosure describes restorative moment detection based on temperature and heart rate data, this is not to be regarded as a limitation of the present disclosure, unless noted otherwise herein. In this regard, the system 200 may be configured to identify restorative moments based on any number or combination of physiological parameters including, but not limited to, temperature data, heart rate data, HRV data, movement data (e.g., acceleration, activity, trainings/workouts), mediation time, MET data, sleep data, galvanic skin temperature data, and the like.

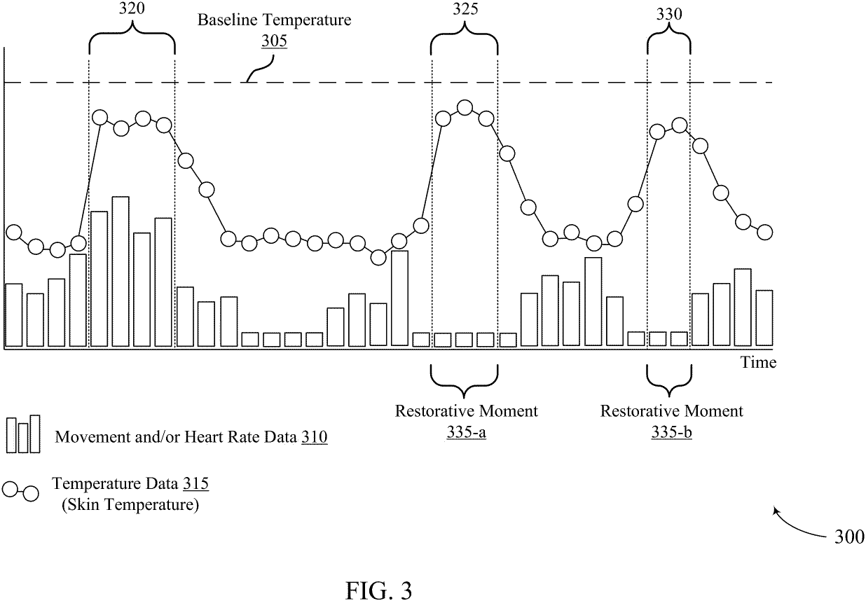

Restorative moments may be further shown and described with reference to FIG. 3

FIG. 3 illustrates an example of a timing diagram 300 that supports techniques for identifying restorative moments 335 in accordance with aspects of the present disclosure. The timing diagram 300 may implement, or be implemented by, aspects of the system 100, system 200, or both. For example, in some implementations, the timing diagram 300 indicating a relative timing of restorative moments may be displayed to a user via the GUI 275 of the user device 102, as shown in FIG. 2.

As will be described in further detail herein, the system 200 may be configured to identify restorative moments 335 for a user based on the user’s temperature and movement/heart rate data (or additional/altemative parameters, as described herein). As such, the timing diagram 300 illustrates a relationship between a user’s temperature data 315 and identified restorative moments 335. Further, there is a direct relationship between a user’s heart rate and movement (e.g., increased movement leads to increased heart rate, and decreased movement leads to decreased heart rate). In this regard, the vertical “bars” illustrated in the timing diagram 300 may be understood to refer to a user’s heart rate (e.g., heart rate data), movement (e.g., movement data), or both. In this regard, the vertical bars illustrated in the timing diagram 300 may be referred to as “movement data 310,” “heart rate data 310,” or both, depending on the physiological parameters being used to identify restorative moments 335. In other words, periods of increased movement data 310 may generally correspond to periods of increased heart rate data 310, and vice versa. In some cases, the system 200 may determine, or estimate, movement data 310 for a user based on heart rate data 310 for the user collected via the ring 104. In other cases, the ring 104 may determine movement data 310 for the user via other sensors, such as accelerometers.

The timing diagram 300 shown in FIG. 3 illustrates a relative timing of restorative moments 335 relative to traditional calendar days. In particular, the timing diagram 300 illustrates restorative moments 335 (e.g., moments when the user may be in a relaxed state) for a user throughout one calendar day or a portion of one calendar day. A restorative moment 335 may be defined as a duration of time that contributes more energy and mental readiness to the user than it consumes from the user. For example, a user may experience a restorative moment 335 when they are resting, meditating, and the like.

The timing diagram 300 may illustrate restorative moments throughout a sleep day, where the sleep day may or may not be aligned with traditional calendar days. In some cases, the timing diagram 300 may illustrate detected restorative moments 335 during a sleep day that aligns with the traditional calendar days, such that the illustrated sleep day runs from midnight to midnight of the respective calendar day. In other cases, the timing diagram 300 may illustrate detected restorative moments 335 during a sleep day that is offset relative to calendar days (e.g., represent a portion of a calendar day). For example, the timing diagram 300 may illustrate detected restorative moments 335 throughout a sleep day that runs from 6:00 pm on a first calendar day to 6:00 pm on a second, subsequent calendar day. In some cases, users may be able to selectively adjust (e.g., via the GUI) a timing of a sleep day (e.g., adjust a relative timing of the timing diagram 300) relative to calendar days so that the restorative moments are aligned with the duration of time that the respective users typically are awake.

In some implementations, the system 200 may “weight” certain time periods as being more likely to exhibit restorative moments 335. For example, users may be more likely to experience restorative moments 335 during the day as compared to early in the morning or late in the evening. In this example, the system 200 may be configured to “weight” data collected during different time periods as being more or less likely to be indicative of a restorative moment 335. In some aspects, the system 200 may weight data as being more or less indicative of restorative moments 335 based on the user’s own historical moment data (e.g., the user’s own circadian rhythm), based on physiological data collected from other users (e.g., user’s that share physiological characteristics with the user), via crowdsourcing means, or any combination thereof.

For example, according to a user’s natural circadian rhythm, the user may be more likely to experience restorative moments 335 between 10:00 am and noon, and may be less likely to experience restorative moments 335 between 3:00 pm and 5:00 pm. In this example, the system 200 may be configured to “weight” physiological data collected between 10:00 am and noon as being more likely to include or indicate restorative moments 335 as compared to physiological data collected between 3:00 pm and 5:00 pm. In some cases, “weighting” physiological data may include inputting weights into a classifier, adjusting thresholds used to identify restorative moments 335, or any combination thereof.

The timing diagram 300 may illustrate a time interval including 1-minute temperature averages and 1-minute heart rate averages over a 36-minute period (e.g., from 11:00 am (17:00) of a calendar day until 11:36 am (17:36) of the same calendar day). The ring 104 of the system 200 may sample the user’s skin temperature (e.g., temperature data 315) every five seconds and store the measurements as 1-minute averages. The timing diagram 300 may include five restorative moments (e.g., minutes) from 11:00 am until 11:36 am, as described below in further detail. The timing diagram 300 may also include four active moments (e.g., minutes) from 11:00 am until 11:36 am, as described below in further detail.

In some cases, the system 200 (e.g., ring 104, user device 106, server 110) may receive physiological data associated with the user from a wearable device 104. The physiological data may include at least heart rate data 310 and temperature data 315. As noted previously herein, the system 200 may utilize additional or alternative parameters to identify restorative moments, including, but not limited to, temperature data, heart rate data, HRV data (e.g., daytime HRV), movement data (e.g., acceleration, activity, trainings/workouts, training Stress Score), mediation time, MET data (e.g., continuous MET levels), sleep data, galvanic skin temperature data, and the like.

The system 200 may identify a baseline temperature 305 associated with the user based on receiving the physiological data. In some examples, the baseline temperature 305 may include a nighttime temperature baseline. In other words, in some implementations, the system 200 may utilize personalized cutoffs or thresholds, in which the user’s daytime temperature data is compared to the user’s nighttime temperature reference/baseline data. In other examples, the system 200 may identify a nighttime temperature baseline for a group of users and identify the baseline temperature 305 associated with the user based on identifying the nighttime temperature baseline. In such cases, the system may determine a nighttime baseline temperature based on average nighttime baseline temperature of a group of people. Determining a baseline temperature 305 for a group of users may be particularly beneficial when identifying restorative moments for new users (e.g., users who recently started wearing a ring 104), as the system 200 may not have sufficient data to determine the user’s own personal baseline temperature. The baseline temperature 305 may be, for example, 35-36.5° C., which may be within a range of the actual core body temperature of the user. When the user is awake, the skin temperature (e.g., temperature data 315) may be lower than the baseline temperature 305. For example, the skin temperature may be between 22-34° C. when the user is awake. The baseline temperature 305 may be used to determine when the user is in a relaxed state (e.g., experiencing a restorative moment 335).

The system 200 may identify a heart rate threshold associated with the user based on receiving the physiological data. In some cases, the heart rate threshold may change throughout the calendar day. For example, the heart rate threshold of the user may change throughout the course of the day according to the user’s circadian rhythm. In such cases, the heart rate threshold may not represent a single heart rate threshold throughout the day. For example, what may be identified as an unexpected heart rate at night, may be an expected heart rate during the day (e.g., a user’s “normal” heart rate at 2:00 pm may be different from the user’s “normal” the heart rate at 4:00 am).

In this regard, the system 200 may be configured to determine “baseline physiological data” (e.g., baseline temperature data, baseline heart rate data) according to the user’s own biological rhythms (e.g., circadian rhythms, ultradian rhythms, seasonal rhythms), and may be configured to compare collected temperature data 315 and/or heart rate data 310 to the determined baseline data. In other words, the user’s baseline physiological data (e.g., baseline temperature 305, heart rate threshold) may be dynamic based on the user’s own biological rhythms. Moreover, thresholds against which collected temperature data and/or heart rate data is compared may be based on the user’s dynamic baseline data/thresholds. For example, in order to identify restorative moments 335, the system 200 may utilize a first heart rate threshold at 2:00 am and a second heart rate threshold at 4:00 pm based on the user’s baseline heart rate data 310 that is a function of the user’s natural circadian rhythm. In other words, the system 200 may be configured to compensate for natural changes in a user’s physiological parameters that are attributable to the user’s natural circadian rhythm (and/or other rhythms). By way of another example, the baseline temperature 305 may vary throughout a 24-hour day based on the user’s natural circadian rhythm (and/or other rhythms). In this regard, the baseline temperature 305 against which a user’s temperature data is compared to identify restorative moments may be different at 2:00 am as compared to 4:00 pm.

In some implementations, the system may identify that the heart rate data 310 may be less than or equal to the heart rate threshold for at least a portion of a time interval (e.g., time interval 330). The system may identify that the temperature data 315 is within a temperature range of the baseline temperature 305 associated with the user for at least the portion of the time interval (e.g., time interval 330). For example, the temperature data 315 (e.g., skin temperature of the user) may occasionally climb close to the baseline temperature 305. In such cases, when the temperature data 315 increases to the baseline temperature 305 while the heart rate data 310 is below the heart rate threshold, the system may determine that the user is in a relaxed state (e.g., experiencing a restorative moment 335-a). Based on the heart rate data 310 being less than or equal to the heart rate threshold and the temperature data 315 being within the temperature range of the baseline temperature 305 for time interval 325, the system 200 may identify a restorative moment 335-a for the time interval 325 that the user is in a relaxed state. As the user relaxes, blood flow may increase through the periphery parts of the body (e.g., fingers and toes). As the blood is spread throughout a larger volume, the blood pressure may decrease, and thus, the heart rate may decrease. The temperature data 315 may increase as increased amounts of warm blood circulate through the periphery parts of the body.

In other implementations, the system may identify that the heart rate data 310 may be greater than the heart rate threshold for at least a portion of a time interval (e.g., time interval 320). The system may identify that the temperature data 315 is within a temperature range of the baseline temperature 305 associated with the user for at least the portion of the time interval (e.g., time interval 320). Based on the heart rate data 310 being greater than the heart rate threshold and the temperature data 315 being within the temperature range of the baseline temperature 305, the system 200 may identify that the user is in a non-relaxed state for the time interval 320. For example, when the heart rate increases as the skin temperature (e.g., temperature data 315) increases, the system 200 may determine that the user is participating in physical activity during time interval 320 (e.g., experiencing an active moment, and therefore not experiencing a restorative moment 335).

In other implementations, the system 200 may identify that the heart rate data 310 may be greater than the heart rate threshold for at least a portion of the time interval (e.g., time interval 320). The system may identify that the temperature data 315 is outside a temperature range of the baseline temperature 305 associated with the user for at least the portion of the time interval (e.g., time interval 320). Based on the heart rate data 310 being greater than the heart rate threshold and the temperature data 315 being outside the temperature range of the baseline temperature 305, the system 200 may identify that the user is in a non-relaxed state. In some cases, the user may have a heart rate that is below the threshold, but the user may also be experiencing a non-relaxed state (e.g., stressed state).

As noted previously herein, the system 200 may identify restorative moments based on temperature data 315 and heart rate data 310 for a user. In additional or alternative cases, the system 200 may utilize movement data 310 for the user to identify restorative moments 335. For example, the physiological data may further include movement data 310. In such cases, the system may identify that the movement data 310 is within a range of a baseline movement associated with the user for at least the portion of the time interval (e.g., time interval 330), and may therefore identify a restorative moment 335-b for the user based on the movement data 310. Identifying the restorative moment 335-b for the time interval 330 may be based on the movement data 310 being within the range of the baseline movement, the temperature data 315 being within a range of the baseline temperature 305, heart rate data 310 being less than a heart rate threshold, or any combination thereof. In other words, the system 200 may leverage temperature data 315, movement data 310, heart rate data 310, or any combination thereof, to identify restorative moments. The baseline movement may be determined based on the user’s average history of movement during the time interval.

For example, in some implementations, the system 200 may identify that the movement data 310 may be less than or equal to a movement threshold for at least a portion of a time interval (e.g., time interval 330). The system may identify that the temperature data 315 is within the temperature range of the baseline temperature 305 associated with the user for at least the portion of the time interval (e.g., time interval 330). Based on the movement data being less than or equal to the movement threshold and the temperature data 315 being within the temperature range of the baseline temperature 305, the system 200 may identify a restorative moment 335-b for the time interval 330 that the user is in a relaxed state. That is, when the temperature data 315 may be close to the baseline temperature 305 while the user may be stationary (e.g., movement data 310 below the threshold), the system 200 may determine that the user is relaxed and experiencing a restorative moment 335-b.

Comparatively, in other implementations, the system 200 may identify that the movement data 310 may be greater than the movement threshold for at least a portion of the time interval 320. The system 200 may identify that the temperature data 315 is within a temperature range of the baseline temperature 305 associated with the user for at least the portion of the time interval 320. Based on the movement data 310 being greater than the movement threshold and the temperature data 315 being within the temperature range of the baseline temperature 305, the system 200 may identify that the user is in a non-relaxed state for the time interval 320. For example, when motion is present (e.g., movement data 310 is above a threshold) as the skin temperature (e.g., temperature data 315) increases, the system 200 may determine that the user is participating in physical activity during time interval 320.

In additional or alternative implementations, the system 200 may identify restorative moments based on changes or trends in physiological parameters such as heart rate data 310 and temperature data 315. In other words, restorative time may be detected when biosignals (e.g., temperature data 315, heart rate data 310) starts to change, and not only when the respective biosignals have achieved or reached s specific value or level. For example, in some cases, an increase in a user’s temperature data 315 and a decrease in a user’s heart rate data 310 may be used to identify a restorative moment 335, even if the absolute values of the temperature data 315 and/or the heart rate data 310 do not satisfy the respective thresholds/baselines (e.g., even if the temperature data 315 is not within a range of the baseline temperature 305). In this regard, the system 200 may be configured to identify a restorative moment 335

In some implementations, the system 200 may additionally or alternatively utilize other physiological parameters to identify restorative moments 335, such as HRV data. For example, in some cases, the physiological data collected by the ring 104 may include HRV data. In such cases, the system 200 may identify that the HRV data is within a range of a baseline HRV associated with the user for at least the portion of the time interval 325, and may therefore identify the restorative moment 335-a based on the HRV data. Identifying the restorative moment 335-a for the time interval 325 may be based on the HRV data being within the range of the baseline HRV, the heart rate data 310 being less than or equal to the heart rate threshold, and the temperature data 315 being within the temperature range of the baseline temperature 305. In other words, the system 200 may identify restorative moments 335 based on temperature data 315, movement data 310, heart rate data 310, HRV data, or any combination thereof.

In some implementations, the system 200 may leverage additional or alternative physiological parameters to identify restorative moments 335, such as galvanic skin response data. For example, the physiological data collected from a user via a ring 104 may include galvanic skin response data. In such cases, the system 200 may identify that the galvanic skin response data is within a range of a baseline galvanic skin response associated with the user for at least the portion of the time interval 330, and may therefore identify a restorative moment 335-b for the time interval 330 based on the galvanic skin response data. Identifying the restorative moment 335-b for the time interval 330 may be based on the galvanic skin response data being within the range of the baseline galvanic skin response, the heart rate data 310 being less than or equal to the heart rate threshold, and the temperature data 315 being within the temperature range of the baseline temperature 305. In some cases, the physiological data may include accelerometer data, metabolic equivalent data (MET), or both. MET may be the amount of oxygen consumed by the user while sitting at rest.

In some implementations, the system 200 may utilize classifiers (e.g., machine learning classifiers) to identify restorative moments 335. In particular, the system 200 may train a classifier to identify restorative moments for a user based on inputted physiological data for the user. For example, in some cases, physiological data collected from a user (e.g., including the heart rate data 310, temperature data 315, movement data 310, HRV data, and/or galvanic skin response data) may be inputted into a classifier (e.g., machine learning classifier), where identifying restorative moments 335 is based on inputting the physiological data into the machine learning classifier (e.g., the classifier is configured to identify restorative moments 335).

In some implementations, user inputs received from a user (e.g., via GUI 275 of the user device 106) may be used to further train a classifier to identify restorative moments. In other words, a user may be able to generate user inputs that may then be used to train the machine learning classifier. For example, the classifier may identify/predict a restorative moment 335 for a user based on received physiological data. Subsequently, the system 200 may prompt the user (e.g., via the GUI 275) to confirm or deny whether the user experienced a restorative moment 335, and the user inputs (e.g., confirmation or denial of a restorative moment 335) may be used to further train the classifier to become more effective at accurately identifying restorative moments 335.

A daytime restorative moment 335 may be estimated based on personalized reference values and physiological data for each individual user. In other words, thresholds and reference values used to determine restorative moments may be individualized for each respective user. For example, the system 200 may use a nighttime average heart rate that may be an example of a recursive average of the last thirty nights with exceptional nights (e.g., because of sickness, alcohol, traveling, etc.) omitted from the average. The system 200 may also use a nighttime temperature deviation that may be an example of a recursive average of the last thirty nights with exceptional nights (e.g., because of sickness, alcohol, traveling, etc.) omitted from the average. The system 200 may also use the daytime continuous skin temperature (e.g., temperature data 315), daytime continuous heart rate data 310, a population based average difference between nighttime and daytime temperatures, a cutoff point for daytime temperature, and a cutoff point for daytime heart rate.

In such cases, the system 200 may identify a restorative moment based on any number of physiological parameters or values, such as a user’s daytime heart rate (HR_{Daytime}), nighttime average heart rate (HR_{NighttimeAvg}), daytime skin temperature (T_{SkinDaytime}), nighttime temperature deviation (T_{NighttimeDeviation}), population-based average between nighttime and daytime temperatures (T_{PopDeviation}), temperature cutoff points (T_{Cutoff}), heart rate cutoff points (HR_{Cutoff} ),and the like. Moreover, the system 200 may be configured to utilize any equation, algorithm, or other mathematical operation to identify restorative moments. For example, the system 200 may be configured to identify a restorative moment for a user if T_{SkinDaytime} + T_{NighttimeDeviation} - T_{PopDeviation} > T_{Cutoff}. Additionally, or alternatively, the system 200 may be configured to identify a restorative moment for a user if HR_{Daytime} -HR_{NighttimeAvg} < HR_{Cutoff}.

In other examples, a daytime restorative moment 335 may be estimated based on personalized chronotype-specific daytime trends. For example, the system 200 may use a nighttime average heart rate that may be an example of a recursive average of the last thirty nights with exceptional nights (e.g., because of sickness, alcohol, traveling, etc.) omitted from the average. The system may also use a nighttime temperature deviation that may be an example of a recursive average of the last thirty nights with exceptional nights (e.g., because of sickness, alcohol, traveling, etc.) omitted from the average. The system 200 may also use the daytime continuous skin temperature (e.g., temperature data 315), daytime continuous heart rate data 310, a population based average difference between nighttime and daytime temperatures, a cutoff point for daytime temperature, and a cutoff point for daytime heart rate.

To determine restorative moments 335, the system 200 may also use continuous daytime MET-level, daytime temperature skin difference, daytime temperature skin difference rates, and daytime difference temperature skin time window. In some cases, the skin temperature (e.g., temperature data 315) may not need to be at a certain level to show that the user is in a relaxed state if the skin temperature begins at a sufficient rate to approach a sufficient temperature. The system 200 may determine the start of the restorative moment window based on using continuous daytime MET-level, daytime skin temperature difference, daytime skin temperature difference rates, and daytime skin temperature difference time windows.

In some implementations, the system 200 may also use training stress score (TSS) MET and training compensation heart rate to identify restorative moments 335. The TSS may be based on the intensity and duration of the workout and indicate how hard the workout was for the user. In some cases, the time after training may be restorative, even though the heart rates may be elevated due to the training. In such cases, the TSS may help determine the post-workout heart rate compensation and the time window that the heart rate may be allowed to be higher so that, relative to time, the compensation decreases. In some cases, the system 200 may use the personalized morning heart rate compensation and the personalized evening heart rate compensation. The biological signals of the user may include a chronological rhythm that may include unique chronotypes. In the evening, the heart rate of the user may be lower and the temperature higher. In such cases, this allows the individual chronotype to be considered in determining a restorative moment 335. For example, the reference heart rate (e.g., heart rate threshold) may not be the average nighttime heart rate, but rather a trend adjusted to the time of day that a lower heart rate may be present in the evening as compared to the user’s heart rate in the morning.

In some aspects, the system 200 may identify restorative moments 335 relative to trainings or workouts. In other words, identified trainings (e.g., TSS) may be used to adjust an amount of identified restorative moments 335 and/or how restorative moments 335 are identified. For example, after a hard workout, a user may exhibit an elevated heart rate (e.g., high heart rate data 310) which may preempt identification of a restorative moment 335 even if the user could otherwise be experiencing a restorative moment 335 after the workout. As such, in some cases, the system 200 may adjust thresholds, baselines, algorithms, etc., used to identify restorative moments 335 following a training or workout. For example, the system 200 may adjust thresholds (e.g., baseline temperature 305, baseline heart rate) for a certain time period following completion of a training or workout to enable the system to identify restorative moments 335 following the training/workout that would otherwise not be identified as restorative moments but for the adjusted thresholds/baselines.

The system 200 may be configured to detect multiple restorative moments 335 within a day. Moreover, the system 200 may be configured to adjust Sleep Scores and Readiness Scores based on restorative moments 335 detected throughout the day. In addition to supporting a diverse group of users, being able to detect restorative moments 335 outside of the primary sleep period may provide more accurate health information to the users, and may improve business-to-business (B2B) use cases, such as illness detection initiatives. Moreover, as noted previously herein, the detection of “restorative moments” for a user may enable the system 200 to determine a more complete and accurate picture of the user’s overall restfulness and recovery (e.g., more accurate Readiness Scores). As such, by enabling more complete and accurate Readiness Scores, techniques described herein may enable the system 200 to provide improved insights and guidance to the user that better correlate to the user’s overall recovery.

For the purposes of the present disclosure, the terms “restorative minutes,” “restorative moments,” “restorative time,” and like terms, may be used interchangeably. In some cases, the system 200 (e.g., user device 106, server 110) may be configured to receive data collected from a user via the ring 104, and identify periods that the user is determined to be in a relaxed state. In some cases, any heart rate data 310 that is less than or equal to a heart rate threshold and any temperature that is within a temperature range of a baseline temperature 305 may be classified as a restorative minute. However, it is contemplated herein that other thresholds may be used to classify restorative minutes.

In some aspects, restorative moment 335 detection allows users to get “credit” for being in a relaxed state outside of their sleeping period. Restorative moments 335 that are outside of the sleep period and which satisfy some threshold time duration may be referred to (e.g., classified) as restorative moments 335, and their contribution to Readiness Scores and/or other scores (e.g., Sleep Scores) may be calculated. Data collected by the ring 104 during a restorative moment 335 may be used to generate the data (via the ring 104), including the score deltas, hypnograms, sleep stages, heart rate graphs, HRV graphs, and the like. However, the score impact (e.g., impact on Sleep Score and Readiness Score) from restorative moments 335 may be relatively small. Moreover, if a pre-existing score (e.g., initial Sleep Score, initial Readiness Score) is relatively high, detected restorative moments 335 may have a relatively small effect on the overall scores (if any). For the average user, the changes introduced by restorative moment 335 detection may be relatively minor.

For example, referring to the system 200 illustrated in FIG. 2, the ring 104 may be worn by a user and may collect data associated with the user throughout a day. The ring 104 may collect data (e.g., temperature, heart rate, HRV, movement, galvanic skin response) and transmit collected data to the user device 106. In some cases, the user device 106 may forward (e.g., relay, transmit) the data received from the ring 104 to the servers for processing. Additionally, or alternatively, the user device 106 and/or the ring 104 may perform processing on the collected data.

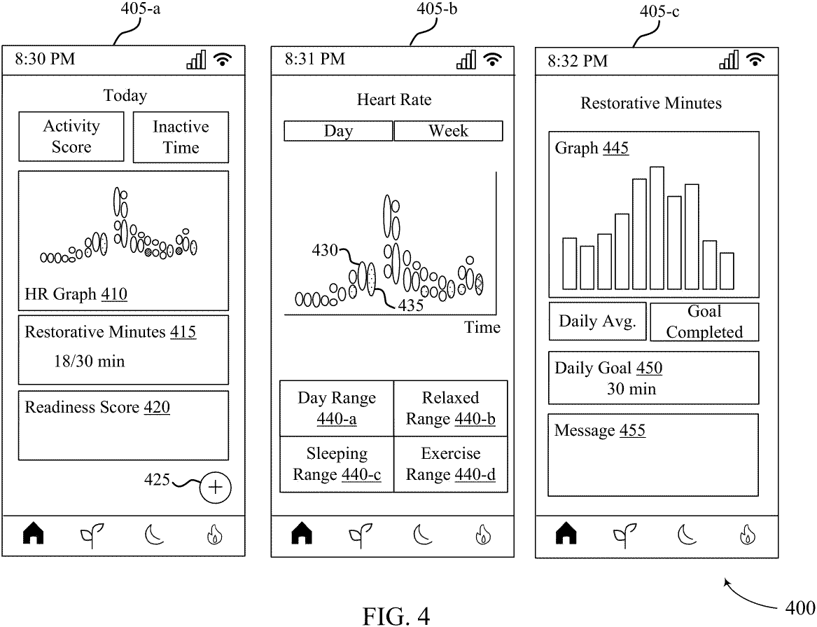

Continuing with the same example, the ring 104, the user device 106, the servers 110, or any combination thereof, may detect a restorative moment 335 based on the collected data. Upon detecting the restorative moment 335, the servers 110 may transmit an indication of the detected restorative moment 335 to the user device 106. Alternatively, in cases where the user device 106 performs data processing, the user device 106 may generate the indication of the detected restorative moment 335. In this example, the next time the user opens the wearable application 250, an indication of the detected restorative moment 335 may be presented to the user via the GUI 275 of the user device 106. This may be further understood with reference to FIG. 4.

In some implementations, the system 200 may utilize subjective data or contextual “tags” to identify trends and more efficiently identify restorative moments 335 for a user. In particular, a user may “tag” certain events using the user device 106, and the system 200 may identify whether respective tags are more or less likely to precede or follow restorative time. Subjective data or “tags” may indicate certain events, environmental factors, life events, or feelings, such as alcohol/caffeine consumption, food consumption, stress/anxiety, an exam, a doctor’s appointment, etc. These tags may be used to identify bigger-picture insights and trends used to identify restorative moments 335. For example, based on contextual tags input by the user, the system 200 may determine that the user is less likely to experience restorative time in the hours after consuming alcohol. By way of another example, the system 200 may utilize historical weather data (e.g., from a “weather” application executable by the user device 106) to determine that the user is less likely to experience restorative time in high humidity or low-pressure conditions.

In some implementations, the system 200 may leverage restorative moment trends to provide tailored guidance or insights to the user. For example, if the system 200 determines that the user is less likely to experience restorative moments 335 on days that the user consumes caffeine, the system 200 may display a message that states “It looks like you are not fully recovered. Try to reduce or eliminate caffeine consumption today to improve your restorative time and recovery.”

In some implementations, the system 200 may calculate a target quantity of restorative moments for each user (e.g., a “daily goal” for restorative time). The target may be calculated on historical physiological data/restorative moment data collected from the user, based on historical data of other users (e.g., other users that share physiological characteristics with the user), and the like. For example, the system 200 may determine that a first user should strive to achieve X number of restorative moments each day, where a second user should strive to achieve Y number of restorative moments each day.

In some aspects, the target quantity of restorative moments for each user may be calculated based on each user’s goals or training programs. For example, a first user with a health goal to reduce stress may result in a different calculated target quantity of restorative moments as compared to a second user that is engaging in a marathon training program. Moreover, in some aspects, the system 200 may provide tailored guidance to each user based on their target quantity of restorative moments (e.g., messages indicating that the user is above/below their target). In this regard, the system 200 may be configured to provide tailed messaging and guidance to help each user achieve their target quantity of restorative moments in order to achieve their health goals or engage in a certain health/training program (e.g., tailored guidance to reduce stress levels, or tailored guidance to improve marathon training).

FIG. 4 illustrates an example of a GUI 400 that supports techniques for identifying restorative moments in accordance with aspects of the present disclosure. The GUI 400 may implement, or be implemented by, aspects of the system 100, system 200, timing diagram 300, or any combination thereof. For example, the GUI 400 may include an example of the GUI 275 included within the user device 106 illustrated in FIG. 2.

The GUI 400 illustrates a series of application pages 405 that may be displayed to the user via the GUI 400 (e.g., GUI 275 illustrated in FIG. 2). The server 110 of system 200 may cause the GUI 400 of the user device 106 (e.g., mobile device) to display an indication of the restorative moment (e.g., via application page 405-a, 405-b, or 405-c). In such cases, the system 200 may output the restorative moment on the GUI 275 of the user device 106 to indicate that the user is in a relaxed state.

Continuing with the example above, upon identifying a restorative moment, the user may be presented with the application page 405-a upon opening the wearable application 250. As shown in FIG. 4, the application page 405-a may display an indication that a restorative moment (e.g., restorative minute) was detected via restorative moment card 415. In such cases, the application page 405-a may include a restorative moment card 415 on the home page. Using the restorative moment card 415, the user may identify how many restorative moments the user experienced throughout the day and how many more restorative moments may be needed to reach the daily goal. For example, as shown in application page 405-a, the user may have experienced eighteen restorative moments out of their daily goal of thirty restorative moments.

In some aspects, the system 200 may calculate a “daily goal” or “target” of restorative moments for each respective user (e.g., “target restorative moments”). For example, as shown in the restorative moment card 415, the system 200 may have calculated a daily goal of thirty restorative moments/minutes for the user. The system 200 may calculate daily restorative moment goals for each user based on any number of parameters, including the user’s Sleep Score, Readiness Score, historical restorative moments, determined correlations between restorative moments and Sleep Score/Readiness Score, health goals for the user (e.g., a “reduce stress” health goal), health/training programs that the user is participating in (e.g., a “marathon training” program), and the like. For example, in cases where a user experienced poor sleep that resulted in lower Sleep and Readiness Scores, the system 200 may determine that the user may require additional rest, and may therefore increase the user’s daily goal of restorative moments for that sleep day relative to some average for the user in order to compensate for the low Sleep/Readiness Scores. Additionally, or alternatively, the system 200 may be configured to derive a target number of restorative moments based on an average quantity of restorative moments that the user has previously successfully achieved (e.g., quantity of restorative moments experienced during previous sleep days), particularly if the previous quantity of restorative moments resulted in better Sleep/Readiness Scores the following day. In other words, the system 200 may determine a correlation or relationship between increased restorative moments and increased Sleep/Readiness Scores, and may determine the user’s daily goal for restorative moments based on the correlation.

Additionally, or alternatively, the system 200 may be configured to calculate the “daily goal” of restorative moments for a user (e.g., “target restorative moments”) based on data associated with other users. For example, the system 200 may be configured to calculate a daily goal of restorative moments for a user based on determined daily restorative moments goals for other users (e.g., groups of other users that share one or more demographic characteristics with the user such as age, gender, historical activity, historical Sleep/Readiness Scores). For instance, the system 200 may identify a set of other users with similar Sleep Scores and/or Readiness Scores, may determine calculated target restorative moments for each of the set of users, and may determine/estimate a target restorative moment for the user based on the target restorative moments across the set of users. In other words, the system 200 may be configured to “crowdsource” a determined daily goal of restorative moments for users. Such techniques may be particularly useful in cases where the system 200 does not have sufficient information for a user to calculate a daily restorative moments goal for the user, such as in cases where the user is a new user.

Moreover, the application page 405-a may prompt the user to confirm or dismiss the restorative moment (e.g., confirm/deny whether the system 200 correctly determined that the user was in a relaxed state). Additionally, in some implementations, the application page 405-a may display one or more scores (e.g., Sleep Score, Readiness Score 420) for the user for the respective day (e.g., respective sleep day).

In cases where the user dismisses the prompt on application page 405-a, the prompt may disappear, and the data from the (incorrectly) determined restorative moment may not be used to update the user’s future Sleep and Readiness Scores (e.g., Readiness Score 420 for the following day). Conversely, upon confirming the identified restorative moment on application page 405-a, the GUI 400 may display application page 405-c. The application page 405-c may indicate one or more parameters of the restorative moment, including a time (e.g., time of day) that the restorative moment was detected, a duration of the restorative moment, and the like. In some cases, the user may only be able to confirm the restorative moment within the sleep day that the restorative moment was detected. In other words, in some implementations, “unconfirmed” restorative moments may not be confirmed/added the next sleep day. For example, in the event a detected restorative moment goes unconfirmed before the end of the respective sleep day, the restorative moment may be categorized as a “rest period,” which may or may not be used to affect a user’s score for that respective sleep day.

As shown in FIG. 4, the application page 405-a may display a restorative moment card 415 such as a “reward card” or “restorative moment confirmation card” that indicates that the restorative moment has been recorded. In some implementations, upon confirming that the restorative moment is valid, the restorative moment may be recorded/logged in an activity log for the user for the respective sleep day. Moreover, in some cases, the restorative moment may be used to update (e.g., modify) one or more scores associated with the user (e.g., Sleep Score, Readiness Score 420). That is, data associated with the restorative moment may be used to update the scores for the user for the following sleep day after which the restorative moment was detected.

The server 110 of system 200 may update a Readiness Score 420 associated with the user based on identifying the restorative moment. For example, the server 110 of system 200 may detect restorative moments to calculate a Readiness Score 420 for the next day. In such cases, the restorative moment may be identified within a first sleep day associated with a first Readiness Score 420 for the user. In some implementations, updating the Readiness Score 420 of the user may include updating a second Readiness Score 420 for the user where the second Readiness Score 420 is associated with a second sleep day that is subsequent to the first sleep day. For example, the updated Readiness Score 420 may be for the following sleep day rather than the current sleep day.

In some implementations, the system 200 may be configured to log, record, or otherwise recognize data associated with a restorative moment without explicit confirmation from a user. For example, in some cases, the system may identify a restorative moment with a sufficient degree of precision, accuracy, or reliability (e.g., probability of a detected restorative moment satisfying some threshold). In such cases, the system 200 may log or otherwise record the restorative moment without displaying a prompt to a user and/or receiving an explicit confirmation from the user. The server 110 of system 200 may receive, via the user device 106 and in response to identifying the restorative moment, a confirmation of the restorative moment.

As noted previously herein, the user device 106 and/or servers 110 of the system 200 may be configured to record (e.g., store) and/or update scores associated with the user using the restorative moment. If the restorative moment is detected before the primary sleep period for the respective sleep day (e.g., after an initial Sleep Score and/or Readiness Score 420 has been calculated based on the primary sleep period), the ring 104, user device 106, and/or servers 110 may store the data associated with the restorative moment until the primary sleep period for the sleep day is detected. In this example, the user device 106 and/or servers may be configured to wait until the primary sleep period for the sleep day is detected so that initial scores (e.g., initial Sleep Score, initial Readiness Score 420) for the following day may be calculated based on the restorative moment. Subsequently, the initial scores may be updated based on the data for the restorative moment.

In some implementations, both the changes to the user’s overall Sleep and Readiness Scores 420, as well as the changes to each of the respective contributing factors, may be displayed to the user via the application page 405-a. In other implementations, only the changes to the user’s overall Sleep and Readiness Scores 420 as a result of the restorative moment may be displayed, where the changes to the respective contributing factors may not be displayed, or highlighted. For example, in some cases, the application page 405-a may display the updated values for each of the contributing factors used to calculate the updated Sleep and Readiness Scores 420, but may not display the previous contributing factors that were used prior to the restorative moment. In other words, the application page 405-a may display the updated values for the contributing factors, but may not indicate how the restorative moment changed the respective values.

The server of system may receive, via the user device, an indication of a relaxed moment, an indication of an emotional state associated with the user, or both, via input 425. In some cases, identifying the restorative moment may be based on receiving the indication of the relaxed moment, the indication of the emotional state, or both. For example, the server of system may receive user input, via input 425, on how the user feels or when the users take a “moment.” A “moment” may be an example of when the user mediates and what happens to their temperature and heart rate in response to taking a “moment.”

The restorative moments may be used as a metric to assess how relaxed the user may be while taking a “moment.” For example, the user may experience a 30-minute “moment” session via input 425. After the session has ended, the user may view the results of their moment session. By introducing the restorative moments into the calculation, the user may view heart rate, HRV, skin temperature, and restorative moments during the moment session. For example, the user may receive eighteen restorative minutes during the thirty minute moment session. By analyzing the restorative moment metric over different moment sessions, the user may be able to find out the session types and behaviors (e.g., breathing patterns) that lead to deepest relaxation and increased quantity of restorative minutes.

Application page 405-a may display heart rate graph 410. The heart rate graph 410 may include a visual representation of how the user’s heart rate reacted to different events and activities (e.g., exercise, sleep, rest, etc.). The heart rate graph 410 may also indicate if the user experienced a restorative moment and at what time the restorative moment occurred. Continuing with reference to FIG. 4, a user may be able to select the heart rate graph 410 on the application page 405-a in order to view details associated with the heart rate and recorded restorative moment, as shown in application page 405-b (“heart rate modal”). In other words, tapping on the heart rate graph 410 shown on application page 405-a may cause the GUI 400 to display application page 405-b so that the user may quickly and easily view the impact of the restorative moment on the heart rate of the user. The application page 405-b may include a modal view including details for the heart rate. For example, as shown in FIG. 4, the application page 405-b may illustrate details for the heart rate, including a quantity of restorative moments, a duration of the restorative moment, a timeline for the restorative moment, a graph illustrating the user’s movement throughout the restorative moment, and the like.

The server 110 of system 200 may cause the GUI 400 of the user device 106 to display an indication of the heart rate data on application page 405-b. In some implementations, the server 110 of system 200 may cause the GUI 275 of the user device 106 to display an indication of the movement data. For example, application page 405-b may display colored blocks 430 and 435 within the heart rate graph 410 that show the heart rate range measured during the restorative moments.

The colored block 430 may be an example of a heart rate during a non-restorative moment, and colored block 435 may be an example of a heart rate during a restorative moment. The server 110 of the system 200 may measure the user’s daytime heart rate and display the heart rates via the heart rate graph 410 to be able to highlight the user’s heart rate during restorative moments. Application page 405-b may also include a day heart rate range 440-a, a relaxed heart rate range 440-b, a sleeping heart rate range 440-c, and an exercise heart rate range 440-d. The individual ranges may be on a per-hour basis. The heart rate graph 410 may also indicate a quantity of restorative moments/instances based on a quantity of colored blocks 435. By using the heart rate graph 410 displayed on application page 405-b, the user may readily identify how many restorative moments the user experienced during the day, when the relaxed moments occurred, and how the user’s heart rate reacted to the restorative moments based on the presence of colored blocks 435.

In some implementations, the system 200 may be configured to identify moments or time intervals in which the user experiences stress (e.g., acute stress, or long term/cumulative stress) relative to identified restorative moments. In some implementations, the respective graphs shown in the application pages 405-1, 405-b, and 405-c may display identified stressful moments in addition to identified restorative moments. For example, the heart rate graph illustrated in application page 405-b may include different colored blocks that are used to indicate restorative moments and stressful moments, respectively. In some implementations, identified restorative moments may be used as an input to an algorithm, classifier, or other technique that is used to identify and track acute and long term stress experienced by a user., and may therefore be leveraged for stress management and detection use cases.

Continuing with reference to FIG. 4, a user may be able to select the restorative moment card 415 on the application page 405-a in order to view details associated with the recorded restorative moment, as shown in application page 405-c (“details modal”). In other words, tapping on the restorative moment card 415 shown on application page 405-a may cause the GUI 400 to display application page 405-c so that the user may quickly and easily view the impact of the restorative moment. The application page 405-c may include a modal view including details for the restorative moment. For example, as shown in FIG. 4, the application page 405-c may illustrate details for the restorative moment, including a duration of the restorative moment, a timeline for the restorative moment, a hypnogram of the restorative moment (with restorative moment start and end times as timestamps), a graph 445 illustrating the user’s movement throughout the restorative moment, and the like. Additionally, or alternatively, the application page 405-c may display an effect of the restorative moment on the user’s scores (e.g., changes to Sleep Score and/or Readiness Score), and graphs of other biometric indicators throughout the restorative moment (e.g., graph illustrating the user’s heart rate throughout the course of the restorative moment, graph illustrating the user’s temperature throughout the course of the restorative moment, graph illustrating the user’s HRV throughout the course of the restorative moment).

Upon selecting the restorative moment card 415 on application page 405-a, the GUI 400 may display application page 405-c. Application page 405-c may display a trend associated with the restorative moments over time. The application page 405-c may include a daily goal 450 for restorative moments. The user may modify the daily goal 450 for total duration throughout the day of restorative moments. In some cases, the daily goal 450 may be displayed and include a target for accumulative restorative moments/minutes throughout the day. Application page 405-c may display graph 445. Graph 445 may display a quantity and duration of restorative moments for each day. In such cases, the user may readily identify which days include the least restorative moments and the most restorative moments.

In some aspects, the GUI 400 may illustrate all restorative moments for a user within a given sleep day. In some cases, after confirming a restorative moment, a user may be able to edit or delete the restorative moment. For example, application page 405-c may enable the user to delete the restorative moment, or modify characteristics of the restorative moment. For instance, a user may be able to adjust a duration or time of the restorative moment. Additionally, or alternatively, a user may be able to input (e.g., via the GUI 400 of the user device) feedback regarding the restorative moment. Feedback could include how the user feels after the restorative moment (e.g., rested, rejuvenated, groggy, lethargic). Feedback could be input as narrative descriptions typed by the user, or via selections of pre-defined feedback options. In some implementations, the user device and/or the servers may be configured to utilize feedback entered by the user to determine the effect of restorative moment, and to calculate updates to scores for the user.

After detecting/confirming a restorative moment, the data from the restorative moment may be used to update/modify at least a subset of the factors/contributors used to calculate the user’s overall Readiness Score. By way of another example, the data from the restorative moment may be used to update at least a subset of the factors for the Readiness Score (e.g., subset of sleep, sleep balance, heart rate, HRV balance, recovery index, temperature, activity, activity balance). Subsequently, upon updating the subset of individual factors, the updated factors may be used to update the user’s overall Readiness Score. In some cases, there may be factors for the Readiness Score that may not be affected by a detected restorative moment. For example, in some cases, the recovery index, latency, efficiency, activity, and/or activity balance factors may not be affected/updated by a detected restorative moment.

The server of system may cause the GUI 400 of the user device to display a message 455 associated with the identified restorative moment. The user device may display recommendations and/or information associated with the restorative moment via message 455. As noted previously herein, a restorative moment may be beneficial to a user’s overall health. In some implementations, the user device 106 and/or servers 110 may generate alerts (e.g., messages 455, insights) associated with the restorative moment that may be displayed to the user via the GUI 400 (e.g., application page 405-c). In particular, messages 455 generated and displayed to the user via the GUI 400 may be associated with one or more characteristics (e.g., time of day, duration, success metric) of the detected restorative moment. For example, the message 455 may include a time of day that the restorative moment was identified, a duration of the restorative moment, a success metric associated with the restorative moment, a recommended duration for future restorative moments, a quantity of restorative moments identified, or a combination thereof. In some cases, the message 455 may display a recommendation of how to adjust their lifestyle in the days with lower restorative moments to achieve higher restorative moments.

In some aspects, a “success metric” may indicate whether, or to what extent, the restorative moment positively or negatively affected the user’s overall Readiness Score, individual contributors associated with the Readiness Score, and the like. In particular, higher success metrics may indicate that the restorative moment had an overall positive benefit to the user’s health (e.g., net increase in Sleep Scores, Readiness Scores, individual contributing factors, or any combination thereof), whereas lower success metrics may indicate that the restorative moment had a negligible or overall negative effect to the user’s health (e.g., net decrease in Sleep Scores, Readiness Scores, individual contributing factors, or any combination thereof). In this regard, the system may be configured to display messages 455/insights to the user in order to facilitate effective, healthy patterns for the user.

For example, server 110 of the system 200 may cause the user device 106 to display a message 455 (e.g., insight) that informs the user as to whether the timing of the restorative moment is expected to have a positive/negative impact, whether the duration of the restorative moment is expected to have a positive/negative impact, and the like. In some cases, the messages 455 displayed to the user via the GUI 400 of the user device 106 may indicate how the restorative moment affected the overall scores (e.g., overall Readiness Score) and/or the individual contributing factors. For example, a message may indicate “Your restorative moment improved your Readiness Score, nice going! What type of activity would help you stay focused for the rest of the day?” or “Your increased amount of relaxation in the evening has improved your sleep quality. Keep it up!”

In cases where the timing/duration of the restorative moment was not optimal, the messages 455 may provide suggestions for the user to change their restorative moment patterns in order to improve their general health. For example, the message may indicate “You gave your body a nice break! Keep taking it easy for the rest of the day, but try to avoid extra restorative moments so that you’re ready for a good night’s sleep,” or “How are you feeling after your restorative moment? Sometimes we need all the downtime we can get, but try to resist taking a restorative moment too close to bedtime to make sure that you’re ready for the most important sleep period of the day.”

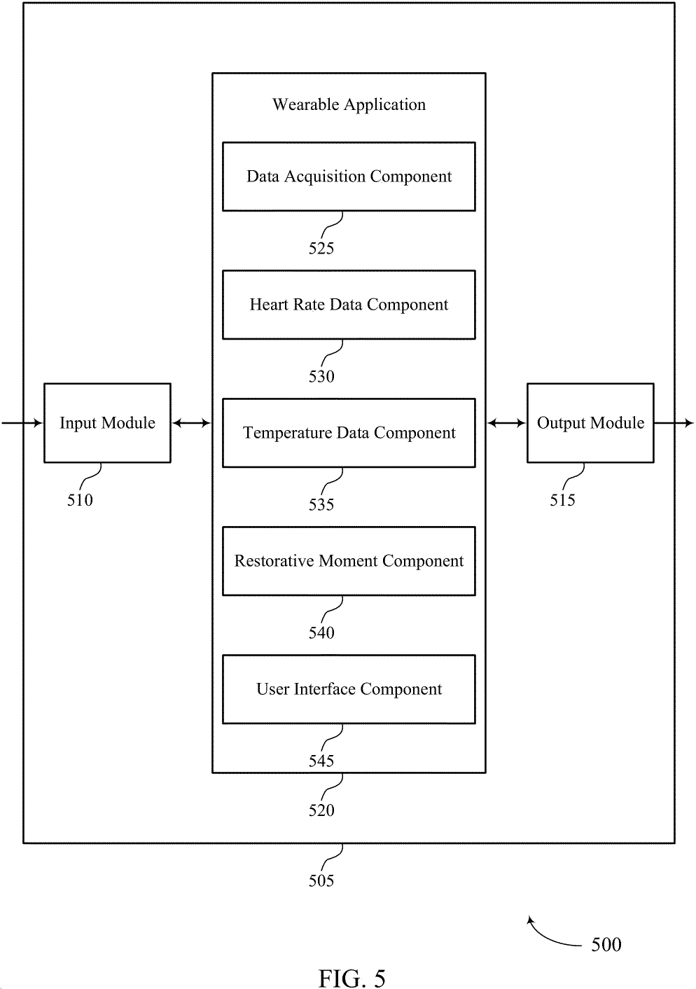

FIG. 5 shows a block diagram 500 of a device 505 that supports techniques for identifying restorative moments in accordance with aspects of the present disclosure. In some implementations, the device 505 may include an example of a user device 106, as described with reference to FIGS. 1-4. The device 505 may include an input module 510, an output module 515, and a wearable application 520. The device 505 may also include a processor. Each of these components may be in communication with one another (e.g., via one or more buses).

The input module 510 may provide a means for receiving information such as packets, user data, control information, or any combination thereof associated with various information channels (e.g., control channels, data channels, information channels related to illness detection techniques). Information may be passed on to other components of the device 505. The input module 510 may utilize a single antenna or a set of multiple antennas.

The output module 515 may provide a means for transmitting signals generated by other components of the device 505. For example, the output module 515 may transmit information such as packets, user data, control information, or any combination thereof associated with various information channels (e.g., control channels, data channels, information channels related to illness detection techniques). In some examples, the output module 515 may be co-located with the input module 510 in a transceiver module. The output module 515 may utilize a single antenna or a set of multiple antennas.

For example, the wearable application 520 may include a data acquisition component 525, a heart rate data component 530, a temperature data component 535, a restorative moment component 540, a user interface component 545, or any combination thereof. In some examples, the wearable application 520, or various components thereof, may be configured to perform various operations (e.g., receiving, monitoring, transmitting) using or otherwise in cooperation with the input module 510, the output module 515, or both. For example, the wearable application 520 may receive information from the input module 510, send information to the output module 515, or be integrated in combination with the input module 510, the output module 515, or both to receive information, transmit information, or perform various other operations as described herein.

The wearable application 520 may support automatically detecting restorative moments in accordance with examples as disclosed herein. The data acquisition component 525 may be configured as or otherwise support a means for receiving physiological data associated with a user from a wearable device, the physiological data comprising at least heart rate data and temperature data. The heart rate data component 530 may be configured as or otherwise support a means for identifying that the heart rate data is less than or equal to a heart rate threshold for at least a portion of a time interval. The temperature data component 535 may be configured as or otherwise support a means for identifying that the temperature data is within a temperature range of a baseline temperature associated with the user for at least the portion of the time interval. The restorative moment component 540 may be configured as or otherwise support a means for identifying a restorative moment for the time interval that the user is in a relaxed state based at least in part on the heart rate data being less than or equal to the heart rate threshold and the temperature data being within the temperature range of the baseline temperature. The user interface component 545 may be configured as or otherwise support a means for causing a GUI of a user device to display an indication of the restorative moment.

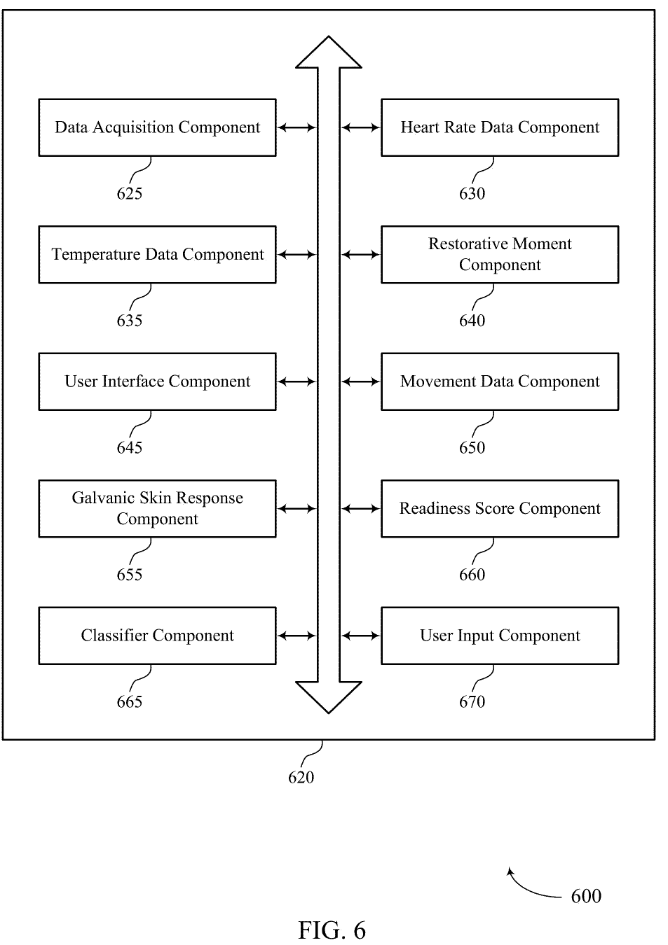

FIG. 6 shows a block diagram 600 of a wearable application 620 that supports techniques for identifying restorative moments in accordance with aspects of the present disclosure. The wearable application 620 may be an example of aspects of a wearable application or a wearable application 520, or both, as described herein. The wearable application 620, or various components thereof, may be an example of means for performing various aspects of techniques for identifying restorative moments as described herein. For example, the wearable application 620 may include a data acquisition component 625, a heart rate data component 630, a temperature data component 635, a restorative moment component 640, a user interface component 645, a movement data component 650, a galvanic skin response component 655, a Readiness Score component 660, a classifier component 665, a user input component 670, or any combination thereof. Each of these components may communicate, directly or indirectly, with one another (e.g., via one or more buses).

The wearable application 620 may support automatically detecting restorative moments in accordance with examples as disclosed herein. The data acquisition component 625 may be configured as or otherwise support a means for receiving physiological data associated with a user from a wearable device, the physiological data comprising at least heart rate data and temperature data. The heart rate data component 630 may be configured as or otherwise support a means for identifying that the heart rate data is less than or equal to a heart rate threshold for at least a portion of a time interval. The temperature data component 635 may be configured as or otherwise support a means for identifying that the temperature data is within a temperature range of a baseline temperature associated with the user for at least the portion of the time interval. The restorative moment component 640 may be configured as or otherwise support a means for identifying a restorative moment for the time interval that the user is in a relaxed state based at least in part on the heart rate data being less than or equal to the heart rate threshold and the temperature data being within the temperature range of the baseline temperature. The user interface component 645 may be configured as or otherwise support a means for causing a GUI of a user device to display an indication of the restorative moment.

In some examples, the temperature data component 635 may be configured as or otherwise support a means for identifying the baseline temperature associated with the user based at least in part on receiving the physiological data, wherein the baseline temperature comprises a nighttime temperature baseline.

In some examples, the physiological data further comprises movement data, and the movement data component 650 may be configured as or otherwise support a means for identifying that the movement data is within a range of a baseline movement associated with the user for at least the portion of the time interval, wherein identifying the restorative moment for the time interval is based at least in part on the movement data being within the range of the baseline movement.

In some examples, the user interface component 645 may be configured as or otherwise support a means for causing the GUI of the user device to display an indication of the heart rate data.

In some examples, the physiological data further comprises HRV data, and the heart rate data component 630 may be configured as or otherwise support a means for identifying that the HRV data is within a range of a baseline HRV associated with the user for at least the portion of the time interval, wherein identifying the restorative moment for the time interval is based at least in part on the HRV data being within the range of the baseline HRV.

In some examples, the physiological data further comprises galvanic skin response data, and the galvanic skin response component 655 may be configured as or otherwise support a means for identifying that the galvanic skin response data is within a range of a baseline galvanic skin response associated with the user for at least the portion of the time interval, wherein identifying the restorative moment for the time interval is based at least in part on the galvanic skin response data being within the range of the baseline galvanic skin response.

In some examples, the Readiness Score component 660 may be configured as or otherwise support a means for updating a Readiness Score associated with the user based at least in part on identifying the restorative moment. In some examples, to support updating the Readiness Score, the Readiness Score component 660 may be configured as or otherwise support a means for updating a second Readiness Score for the user, the second Readiness Score associated with a second sleep day that is subsequent to the first sleep day.

In some examples, the classifier component 665 may be configured as or otherwise support a means for inputting the physiological data into a machine learning classifier, wherein identifying the restorative moment is based at least in part on inputting the physiological data into the machine learning classifier.

In some examples, the user input component 670 may be configured as or otherwise support a means for receiving, via the user device and in response to identifying the restorative moment, a confirmation of the restorative moment.

In some examples, the user input component 670 may be configured as or otherwise support a means for receiving, via the user device, an indication of a relaxed moment, an indication of an emotional state associated with the user, or both, wherein identifying the restorative moment is based at least in part on receiving the indication of the relaxed moment, the indication of the emotional state, or both.

In some examples, the user interface component 645 may be configured as or otherwise support a means for causing the GUI of the user device to display a message associated with the identified restorative moment. In some examples, the message comprises a time of day that the restorative moment was identified, a duration of the restorative moment, a success metric associated with the restorative moment, a recommended duration for future restorative moments, a quantity of restorative moments identified, or a combination thereof.

In some examples, the temperature data component 635 may be configured as or otherwise support a means for identifying a nighttime temperature baseline for a plurality of users. In some examples, the temperature data component 635 may be configured as or otherwise support a means for identifying the baseline temperature associated with the user based at least in part on identifying the nighttime temperature baseline.

In some examples, the wearable device comprises a wearable ring device. In some examples, the wearable device collects the physiological data from the user based on arterial blood flow.

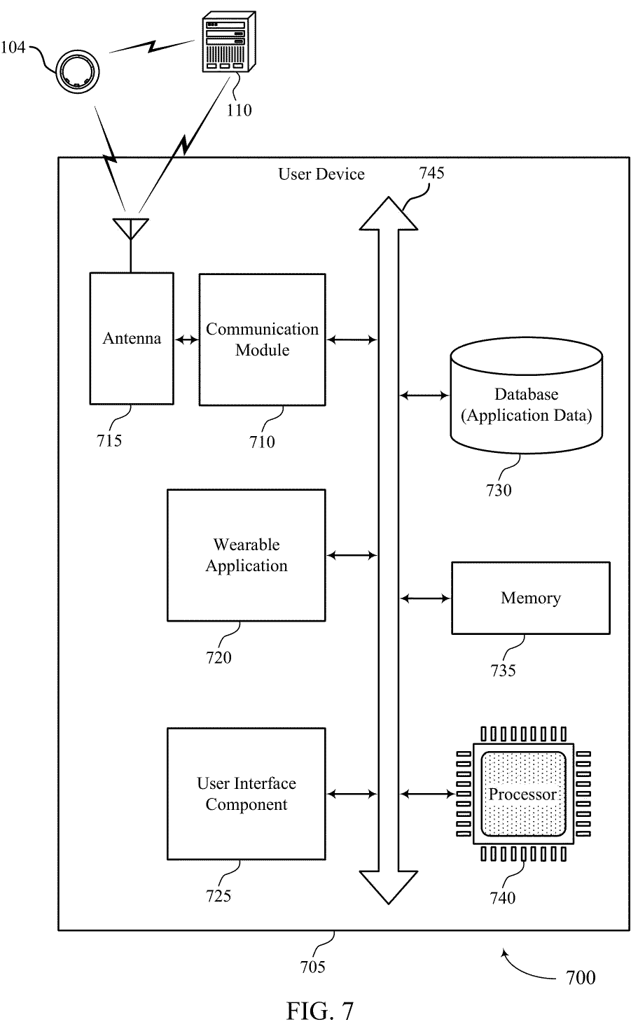

FIG. 7 shows a diagram of a system 700 including a device 705 that supports techniques for identifying restorative moments in accordance with aspects of the present disclosure. The device 705 may be an example of or include the components of a device 505 as described herein. The device 705 may include components for bidirectional data communications including components for transmitting and receiving communications, such as a wearable application 720, a communication module 710, an antenna 715, a user interface component 725, a database (application data) 730, a memory 735, and a processor 740. These components may be in electronic communication or otherwise coupled (e.g., operatively, communicatively, functionally, electronically, electrically) via one or more buses (e.g., a bus 745).

The communication module 710 may manage input and output signals for the device 705 via the antenna 715. The communication module 710 may include an example of the communication module 220-b of the user device 106 shown and described in FIG. 2. In this regard, the communication module 710 may manage communications with the ring 104 and the server 110, as illustrated in FIG. 2. The communication module 710 may also manage peripherals not integrated into the device 705. In some cases, the communication module 710 may represent a physical connection or port to an external peripheral. In some cases, the communication module 710 may utilize an operating system such as iOS®, ANDROID®, MS-DOS®, MS-WINDOWS®, OS/2®, UNIX®, LINUX®, or another known operating system. In other cases, the communication module 710 may represent or interact with a wearable device (e.g., ring 104), modem, a keyboard, a mouse, a touchscreen, or a similar device. In some cases, the communication module 710 may be implemented as part of the processor 740. In some examples, a user may interact with the device 705 via the communication module 710, user interface component 725, or via hardware components controlled by the communication module 710.

In some cases, the device 705 may include a single antenna 715. However, in some other cases, the device 705 may have more than one antenna 715, which may be capable of concurrently transmitting or receiving multiple wireless transmissions. The communication module 710 may communicate bi-directionally, via the one or more antennas 715, wired, or wireless links as described herein. For example, the communication module 710 may represent a wireless transceiver and may communicate bi-directionally with another wireless transceiver. The communication module 710 may also include a modem to modulate the packets, to provide the modulated packets to one or more antennas 715 for transmission, and to demodulate packets received from the one or more antennas 715.

The user interface component 725 may manage data storage and processing in a database 730. In some cases, a user may interact with the user interface component 725. In other cases, the user interface component 725 may operate automatically without user interaction. The database 730 may be an example of a single database, a distributed database, multiple distributed databases, a data store, a data lake, or an emergency backup database.

The memory 735 may include RAM and ROM. The memory 735 may store computer-readable, computer-executable software including instructions that, when executed, cause the processor 740 to perform various functions described herein. In some cases, the memory 735 may contain, among other things, a basic I/O system (BIOS) which may control basic hardware or software operation such as the interaction with peripheral components or devices.

The processor 740 may include an intelligent hardware device, (e.g., a general-purpose processor, a digital signal processor (DSP), a central processing unit (CPU), a microcontroller, an application-specific integrated circuit (ASIC), a field-programmable gate array (FPGA), a programmable logic device, a discrete gate or transistor logic component, a discrete hardware component, or any combination thereof). In some cases, the processor 740 may be configured to operate a memory array using a memory controller. In other cases, a memory controller may be integrated into the processor 740. The processor 740 may be configured to execute computer-readable instructions stored in a memory 735 to perform various functions (e.g., functions or tasks supporting a method and system for sleep staging algorithms).

The wearable application 720 may support automatically detecting restorative moments in accordance with examples as disclosed herein. For example, the wearable application 720 may be configured as or otherwise support a means for receiving physiological data associated with a user from a wearable device, the physiological data comprising at least heart rate data and temperature data. The wearable application 720 may be configured as or otherwise support a means for identifying that the heart rate data is less than or equal to a heart rate threshold for at least a portion of a time interval. The wearable application 720 may be configured as or otherwise support a means for identifying that the temperature data is within a temperature range of a baseline temperature associated with the user for at least the portion of the time interval. The wearable application 720 may be configured as or otherwise support a means for identifying a restorative moment for the time interval that the user is in a relaxed state based at least in part on the heart rate data being less than or equal to the heart rate threshold and the temperature data being within the temperature range of the baseline temperature. The wearable application 720 may be configured as or otherwise support a means for causing a GUI of a user device to display an indication of the restorative moment.

By including or configuring the wearable application 720 in accordance with examples as described herein, the device 705 may support techniques for improved health tracking using data collected by a wearable device. In particular, techniques described herein may be used to detect restorative moments for a given user, which may be used to generate more accurate and comprehensive scores (e.g., Readiness Scores) for the user. By providing a user with a more comprehensive evaluation of their restorative moments, techniques described herein may enable the user to effectively adjust their lifestyle patterns, which may improve the overall health for the user.

The wearable application 720 may include an application (e.g., “app”), program, software, or other component that is configured to facilitate communications with a ring 104, server 110, other user devices 106, and the like. For example, the wearable application 720 may include an application executable on a user device 106 that is configured to receive data (e.g., physiological data) from a ring 104, perform processing operations on the received data, transmit and receive data with the servers 110, and cause presentation of data to a user 102.

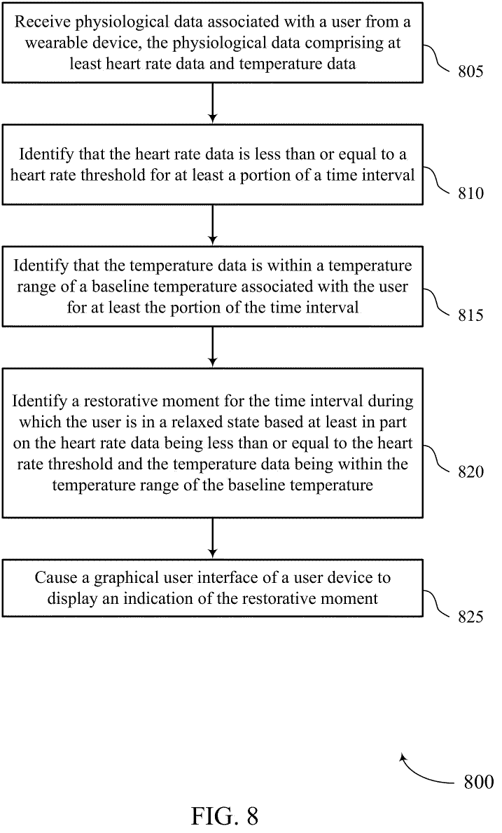

FIG. 8 shows a flowchart illustrating a method 800 that supports techniques for identifying restorative moments in accordance with aspects of the present disclosure. The operations of the method 800 may be implemented by a user device or its components as described herein. For example, the operations of the method 800 may be performed by a user device as described with reference to FIGS. 1 through 7. In some examples, a user device may execute a set of instructions to control the functional elements of the user device to perform the described functions. Additionally or alternatively, the user device may perform aspects of the described functions using special-purpose hardware.

At 805, the method may include receiving physiological data associated with a user from a wearable device, the physiological data comprising at least heart rate data and temperature data. The operations of 805 may be performed in accordance with examples as disclosed herein. In some examples, aspects of the operations of 805 may be performed by a data acquisition component 625 as described with reference to FIG. 6.

At 810, the method may include identifying that the heart rate data is less than or equal to a heart rate threshold for at least a portion of a time interval. The operations of 810 may be performed in accordance with examples as disclosed herein. In some examples, aspects of the operations of 810 may be performed by a heart rate data component 630 as described with reference to FIG. 6.

At 815, the method may include identifying that the temperature data is within a temperature range of a baseline temperature associated with the user for at least the portion of the time interval. The operations of 815 may be performed in accordance with examples as disclosed herein. In some examples, aspects of the operations of 815 may be performed by a temperature data component 635 as described with reference to FIG. 6.

At 820, the method may include identifying a restorative moment for the time interval that the user is in a relaxed state based at least in part on the heart rate data being less than or equal to the heart rate threshold and the temperature data being within the temperature range of the baseline temperature. The operations of 820 may be performed in accordance with examples as disclosed herein. In some examples, aspects of the operations of 820 may be performed by a restorative moment component 640 as described with reference to FIG. 6.

At 825, the method may include causing a GUI of a user device to display an indication of the restorative moment. The operations of 825 may be performed in accordance with examples as disclosed herein. In some examples, aspects of the operations of 825 may be performed by a user interface component 645 as described with reference to FIG. 6.

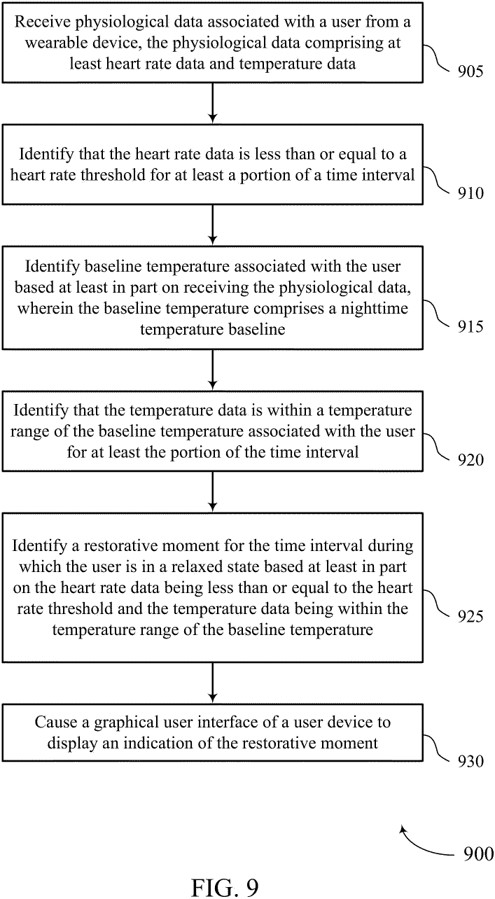

FIG. 9 shows a flowchart illustrating a method 900 that supports techniques for identifying restorative moments in accordance with aspects of the present disclosure. The operations of the method 900 may be implemented by a user device or its components as described herein. For example, the operations of the method 900 may be performed by a user device as described with reference to FIGS. 1 through 7. In some examples, a user device may execute a set of instructions to control the functional elements of the user device to perform the described functions. Additionally or alternatively, the user device may perform aspects of the described functions using special-purpose hardware.

At 905, the method may include receiving physiological data associated with a user from a wearable device, the physiological data comprising at least heart rate data and temperature data. The operations of 905 may be performed in accordance with examples as disclosed herein. In some examples, aspects of the operations of 905 may be performed by a data acquisition component 625 as described with reference to FIG. 6.

At 910, the method may include identifying that the heart rate data is less than or equal to a heart rate threshold for at least a portion of a time interval. The operations of 910 may be performed in accordance with examples as disclosed herein. In some examples, aspects of the operations of 910 may be performed by a heart rate data component 630 as described with reference to FIG. 6.

At 915, the method may include identifying baseline temperature associated with the user based at least in part on receiving the physiological data, wherein the baseline temperature comprises a nighttime temperature baseline. The operations of 915 may be performed in accordance with examples as disclosed herein. In some examples, aspects of the operations of 915 may be performed by a temperature data component 635 as described with reference to FIG. 6.

At 920, the method may include identifying that the temperature data is within a temperature range of the baseline temperature associated with the user for at least the portion of the time interval. The operations of 920 may be performed in accordance with examples as disclosed herein. In some examples, aspects of the operations of 920 may be performed by a temperature data component 635 as described with reference to FIG. 6.

At 925, the method may include identifying a restorative moment for the time interval that the user is in a relaxed state based at least in part on the heart rate data being less than or equal to the heart rate threshold and the temperature data being within the temperature range of the baseline temperature. The operations of 925 may be performed in accordance with examples as disclosed herein. In some examples, aspects of the operations of 925 may be performed by a restorative moment component 640 as described with reference to FIG. 6.

At 930, the method may include causing a GUI of a user device to display an indication of the restorative moment. The operations of 930 may be performed in accordance with examples as disclosed herein. In some examples, aspects of the operations of 930 may be performed by a user interface component 645 as described with reference to FIG. 6.

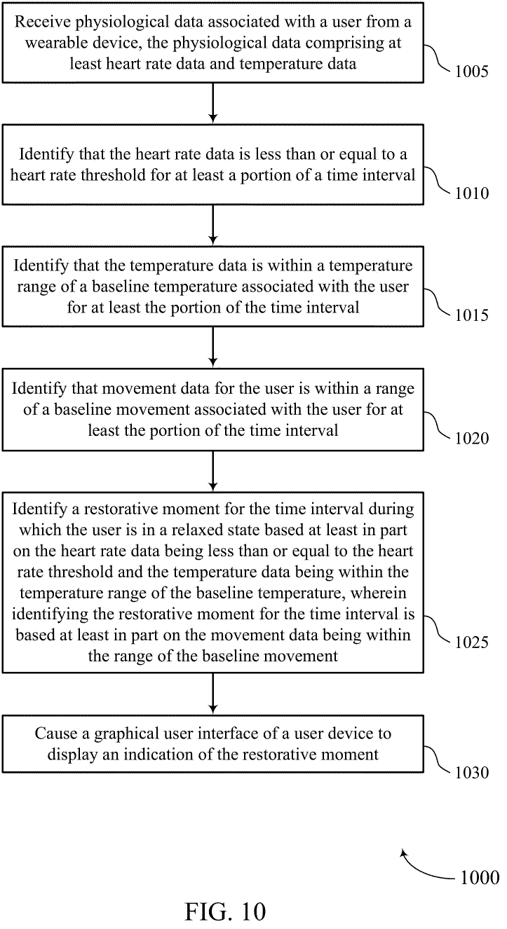

FIG. 10 shows a flowchart illustrating a method 1000 that supports techniques for identifying restorative moments in accordance with aspects of the present disclosure. The operations of the method 1000 may be implemented by a user device or its components as described herein. For example, the operations of the method 1000 may be performed by a user device as described with reference to FIGS. 1 through 7. In some examples, a user device may execute a set of instructions to control the functional elements of the user device to perform the described functions. Additionally or alternatively, the user device may perform aspects of the described functions using special-purpose hardware.

At 1005, the method may include receiving physiological data associated with a user from a wearable device, the physiological data comprising at least heart rate data and temperature data. The operations of 1005 may be performed in accordance with examples as disclosed herein. In some examples, aspects of the operations of 1005 may be performed by a data acquisition component 625 as described with reference to FIG. 6.

At 1010, the method may include identifying that the heart rate data is less than or equal to a heart rate threshold for at least a portion of a time interval. The operations of 1010 may be performed in accordance with examples as disclosed herein. In some examples, aspects of the operations of 1010 may be performed by a heart rate data component 630 as described with reference to FIG. 6.

At 1015, the method may include identifying that the temperature data is within a temperature range of a baseline temperature associated with the user for at least the portion of the time interval. The operations of 1015 may be performed in accordance with examples as disclosed herein. In some examples, aspects of the operations of 1015 may be performed by a temperature data component 635 as described with reference to FIG. 6.

At 1020, the method may include identifying that movement data for the user is within a range of a baseline movement associated with the user for at least the portion of the time interval. The operations of 1020 may be performed in accordance with examples as disclosed herein. In some examples, aspects of the operations of 1020 may be performed by a movement data component 650 as described with reference to FIG. 6.

At 1025, the method may include identifying a restorative moment for the time interval that the user is in a relaxed state based at least in part on the heart rate data being less than or equal to the heart rate threshold and the temperature data being within the temperature range of the baseline temperature, wherein identifying the restorative moment for the time interval is based at least in part on the movement data being within the range of the baseline movement. The operations of 1025 may be performed in accordance with examples as disclosed herein. In some examples, aspects of the operations of 1025 may be performed by a restorative moment component 640 as described with reference to FIG. 6.

At 1030, the method may include causing a GUI of a user device to display an indication of the restorative moment. The operations of 1030 may be performed in accordance with examples as disclosed herein. In some examples, aspects of the operations of 1030 may be performed by a user interface component 645 as described with reference to FIG. 6.

A method for automatically detecting restorative moments is described. The method may include receiving physiological data associated with a user from a wearable device, the physiological data comprising at least heart rate data and temperature data, identifying that the heart rate data is less than or equal to a heart rate threshold for at least a portion of a time interval, identifying that the temperature data is within a temperature range of a baseline temperature associated with the user for at least the portion of the time interval, identifying a restorative moment for the time interval during whichthat the user is in a relaxed state based at least in part on the heart rate data being less than or equal to the heart rate threshold and the temperature data being within the temperature range of the baseline temperature, and causing a GUI of a user device to display an indication of the restorative moment.

An apparatus for automatically detecting restorative moments is described. The apparatus may include a processor, memory coupled with the processor, and instructions stored in the memory. The instructions may be executable by the processor to cause the apparatus to receive physiological data associated with a user from a wearable device, the physiological data comprising at least heart rate data and temperature data, identify that the heart rate data is less than or equal to a heart rate threshold for at least a portion of a time interval, identify that the temperature data is within a temperature range of a baseline temperature associated with the user for at least the portion of the time interval, identify a restorative moment for the time interval during whichthat the user is in a relaxed state based at least in part on the heart rate data being less than or equal to the heart rate threshold and the temperature data being within the temperature range of the baseline temperature, and cause a GUI of a user device to display an indication of the restorative moment.

Another apparatus for automatically detecting restorative moments is described. The apparatus may include means for receiving physiological data associated with a user from a wearable device, the physiological data comprising at least heart rate data and temperature data, means for identifying that the heart rate data is less than or equal to a heart rate threshold for at least a portion of a time interval, means for identifying that the temperature data is within a temperature range of a baseline temperature associated with the user for at least the portion of the time interval, means for identifying a restorative moment for the time interval that the user is in a relaxed state based at least in part on the heart rate data being less than or equal to the heart rate threshold and the temperature data being within the temperature range of the baseline temperature, and means for causing a GUI of a user device to display an indication of the restorative moment.

A non-transitory computer-readable medium storing code for automatically detecting restorative moments is described. The code may include instructions executable by a processor to receive physiological data associated with a user from a wearable device, the physiological data comprising at least heart rate data and temperature data, identify that the heart rate data is less than or equal to a heart rate threshold for at least a portion of a time interval, identify that the temperature data is within a temperature range of a baseline temperature associated with the user for at least the portion of the time interval, identify a restorative moment for the time interval that the user is in a relaxed state based at least in part on the heart rate data being less than or equal to the heart rate threshold and the temperature data being within the temperature range of the baseline temperature, and cause a GUI of a user device to display an indication of the restorative moment.

Some examples of the method, apparatuses, and non-transitory computer-readable medium described herein may further include operations, features, means, or instructions for identifying the baseline temperature associated with the user based at least in part on receiving the physiological data, wherein the baseline temperature comprises a nighttime temperature baseline.

In some examples of the method, apparatuses, and non-transitory computer-readable medium described herein, the physiological data further comprises movement data and the method, apparatuses, and non-transitory computer-readable medium may include further operations, features, means, or instructions for identifying that the movement data may be within a range of a baseline movement associated with the user for at least the portion of the time interval, wherein identifying the restorative moment for the time interval may be based at least in part on the movement data being within the range of the baseline movement.

Some examples of the method, apparatuses, and non-transitory computer-readable medium described herein may further include operations, features, means, or instructions for causing the GUI of the user device to display an indication of the heart rate data.

In some examples of the method, apparatuses, and non-transitory computer-readable medium described herein, the physiological data further comprises HRV data and the method, apparatuses, and non-transitory computer-readable medium may include further operations, features, means, or instructions for identifying that the HRV data may be within a range of a baseline HRV associated with the user for at least the portion of the time interval, wherein identifying the restorative moment for the time interval may be based at least in part on the HRV data being within the range of the baseline HRV.

In some examples of the method, apparatuses, and non-transitory computer-readable medium described herein, the physiological data further comprises galvanic skin response data and the method, apparatuses, and non-transitory computer-readable medium may include further operations, features, means, or instructions for identifying that the galvanic skin response data may be within a range of a baseline galvanic skin response associated with the user for at least the portion of the time interval, wherein identifying the restorative moment for the time interval may be based at least in part on the galvanic skin response data being within the range of the baseline galvanic skin response.

Some examples of the method, apparatuses, and non-transitory computer-readable medium described herein may further include operations, features, means, or instructions for updating a Readiness Score associated with the user based at least in part on identifying the restorative moment.

In some examples of the method, apparatuses, and non-transitory computer-readable medium described herein, updating the Readiness Score may include operations, features, means, or instructions for updating a second Readiness Score for the user, the second Readiness Score associated with a second sleep day which that may be subsequent to the first sleep day.

Some examples of the method, apparatuses, and non-transitory computer-readable medium described herein may further include operations, features, means, or instructions for inputting the physiological data into a machine learning classifier, wherein identifying the restorative moment may be based at least in part on inputting the physiological data into the machine learning classifier.

Some examples of the method, apparatuses, and non-transitory computer-readable medium described herein may further include operations, features, means, or instructions for receiving, via the user device and in response to identifying the restorative moment, a confirmation of the restorative moment.

Some examples of the method, apparatuses, and non-transitory computer-readable medium described herein may further include operations, features, means, or instructions for receiving, via the user device, an indication of a relaxed moment, an indication of an emotional state associated with the user, or both, wherein identifying the restorative moment may be based at least in part on receiving the indication of the relaxed moment, the indication of the emotional state, or both.

Some examples of the method, apparatuses, and non-transitory computer-readable medium described herein may further include operations, features, means, or instructions for causing the GUI of the user device to display a message associated with the identified restorative moment.

In some examples of the method, apparatuses, and non-transitory computer-readable medium described herein, the message comprises a time of day that the restorative moment was identified, a duration of the restorative moment, a success metric associated with the restorative moment, a recommended duration for future restorative moments, a quantity of restorative moments identified, or a combination thereof.

Some examples of the method, apparatuses, and non-transitory computer-readable medium described herein may further include operations, features, means, or instructions for identifying a nighttime temperature baseline for a plurality of users and identifying the baseline temperature associated with the user based at least in part on identifying the nighttime temperature baseline.

In some examples of the method, apparatuses, and non-transitory computer-readable medium described herein, the wearable device comprises a wearable ring device.

In some examples of the method, apparatuses, and non-transitory computer-readable medium described herein, the wearable device collects the physiological data from the user based on arterial blood flow.

It should be noted that the methods described above describe possible implementations, and that the operations and the steps may be rearranged or otherwise modified and that other implementations are possible. Furthermore, aspects from two or more of the methods may be combined.

The description set forth herein, in connection with the appended drawings, describes example configurations and does not represent all the examples that may be implemented or that are within the scope of the claims. The term “exemplary” used herein means “serving as an example, instance, or illustration,” and not “preferred” or “advantageous over other examples.” The detailed description includes specific details for the purpose of providing an understanding of the described techniques. These techniques, however, may be practiced without these specific details. In some instances, well-known structures and devices are shown in block diagram form in order to avoid obscuring the concepts of the described examples.

In the appended figures, similar components or features may have the same reference label. Further, various components of the same type may be distinguished by following the reference label by a dash and a second label that distinguishes among the similar components. If just the first reference label is used in the specification, the description is applicable to any one of the similar components having the same first reference label irrespective of the second reference label.

Information and signals described herein may be represented using any of a variety of different technologies and techniques. For example, data, instructions, commands, information, signals, bits, symbols, and chips that may be referenced throughout the above description may be represented by voltages, currents, electromagnetic waves, magnetic fields or particles, optical fields or particles, or any combination thereof.

The various illustrative blocks and modules described in connection with the disclosure herein may be implemented or performed with a general-purpose processor, a DSP, an ASIC, an FPGA or other programmable logic device, discrete gate or transistor logic, discrete hardware components, or any combination thereof designed to perform the functions described herein. A general-purpose processor may be a microprocessor, but in the alternative, the processor may be any conventional processor, controller, microcontroller, or state machine. A processor may also be implemented as a combination of computing devices (e.g., a combination of a DSP and a microprocessor, multiple microprocessors, one or more microprocessors in conjunction with a DSP core, or any other such configuration).

The functions described herein may be implemented in hardware, software executed by a processor, firmware, or any combination thereof. If implemented in software executed by a processor, the functions may be stored on or transmitted over as one or more instructions or code on a computer-readable medium. Other examples and implementations are within the scope of the disclosure and appended claims. For example, due to the nature of software, functions described above can be implemented using software executed by a processor, hardware, firmware, hardwiring, or combinations of any of these. Features implementing functions may also be physically located at various positions, including being distributed such that portions of functions are implemented at different physical locations. Also, as used herein, including in the claims, “or” as used in a list of items (for example, a list of items prefaced by a phrase such as “at least one of” or “one or more of”) indicates an inclusive list such that, for example, a list of at least one of A, B, or C means A or B or C or AB or AC or BC or ABC (i.e., A and B and C). Also, as used herein, the phrase “based on” shall not be construed as a reference to a closed set of conditions. For example, an exemplary step that is described as “based on condition A” may be based on both a condition A and a condition B without departing from the scope of the present disclosure. In other words, as used herein, the phrase “based on” shall be construed in the same manner as the phrase “based at least in part on.”

Computer-readable media includes both non-transitory computer storage media and communication media including any medium that facilitates transfer of a computer program from one place to another. A non-transitory storage medium may be any available medium that can be accessed by a general purpose or special purpose computer. By way of example, and not limitation, non-transitory computer-readable media can comprise RAM, ROM, electrically erasable programmable ROM (EEPROM), compact disk (CD) ROM or other optical disk storage, magnetic disk storage or other magnetic storage devices, or any other non-transitory medium that can be used to carry or store desired program code means in the form of instructions or data structures and that can be accessed by a general-purpose or special-purpose computer, or a general-purpose or special-purpose processor. Also, any connection is properly termed a computer-readable medium. For example, if the software is transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. Disk and disc, as used herein, include CD, laser disc, optical disc, digital versatile disc (DVD), floppy disk and Blu-ray disc where disks usually reproduce data magnetically, while discs reproduce data optically with lasers. Combinations of the above are also included within the scope of computer-readable media.

The description herein is provided to enable a person skilled in the art to make or use the disclosure. Various modifications to the disclosure will be readily apparent to those skilled in the art, and the generic principles defined herein may be applied to other variations without departing from the scope of the disclosure. Thus, the disclosure is not limited to the examples and designs described herein, but is to be accorded the broadest scope consistent with the principles and novel features disclosed herein.

What is claimed is:

1. A method for automatically detecting restorative moments, comprising:
receiving physiological data associated with a user from a wearable device, the physiological data comprising at least heart rate data and temperature data;
identifying that the heart rate data is less than or equal to a heart rate threshold for at least a portion of a time interval;
identifying that the temperature data is within a temperature range of a baseline temperature associated with the user for at least the portion of the time interval;
identifying a restorative moment for the time interval that the user is in a relaxed state based at least in part on the heart rate data being less than or equal to the heart rate threshold and the temperature data being within the temperature range of the baseline temperature; and
causing a graphical user interface of a user device to display an indication of the restorative moment.

2. The method of claim 1, further comprising:
identifying the baseline temperature associated with the user based at least in part on receiving the physiological data, wherein the baseline temperature comprises a nighttime temperature baseline.

3. The method of claim 1, wherein the physiological data further comprises movement data, the method further comprising:
identifying that the movement data is within a range of a baseline movement associated with the user for at least the portion of the time interval, wherein identifying the restorative moment for the time interval is based at least in part on the movement data being within the range of the baseline movement.

4. The method of claim 1, further comprising:
causing the graphical user interface of the user device to display an indication of the heart rate data.

5. The method of claim 1, wherein the physiological data further comprises heart rate variability data, the method further comprising:
identifying that the heart rate variability data is within a range of a baseline heart rate variability associated with the user for at least the portion of the time interval, wherein identifying the restorative moment for the time interval is based at least in part on the heart rate variability data being within the range of the baseline heart rate variability.

6. The method of claim 1, wherein the physiological data further comprises galvanic skin response data, the method further comprising:
identifying that the galvanic skin response data is within a range of a baseline galvanic skin response associated with the user for at least the portion of the time interval, wherein identifying the restorative moment for the time interval is based at least in part on the galvanic skin response data being within the range of the baseline galvanic skin response.

7. The method of claim 1, further comprising:
updating a readiness score associated with the user based at least in part on identifying the restorative moment.

8. The method of claim 7, wherein the restorative moment is identified within a first sleep day associated with a first readiness score for the user, and wherein updating the readiness score comprises:
updating a second readiness score for the user, the second readiness score associated with a second sleep day that is subsequent to the first sleep day.

9. The method of claim 1, further comprising:
inputting the physiological data into a machine learning classifier, wherein identifying the restorative moment is based at least in part on inputting the physiological data into the machine learning classifier.

10. The method of claim 1, further comprising:
receiving, via the user device and in response to identifying the restorative moment, a confirmation of the restorative moment.

11. The method of claim 1, further comprising:
receiving, via the user device, an indication of a relaxed moment, an indication of an emotional state associated with the user, or both, wherein identifying the restorative moment is based at least in part on receiving the indication of the relaxed moment, the indication of the emotional state, or both.

12. The method of claim 1, further comprising:
causing the graphical user interface of the user device to display a message associated with the identified restorative moment.

13. The method of claim 12, wherein the message comprises a time of day that the restorative moment was identified, a duration of the restorative moment, a success metric associated with the restorative moment, a recommended duration for future restorative moments, a quantity of restorative moments identified, or a combination thereof.

14. The method of claim 1, further comprising:
identifying a nighttime temperature baseline for a plurality of users; and
identifying the baseline temperature associated with the user based at least in part on identifying the nighttime temperature baseline.

15. The method of claim 1, wherein the wearable device comprises a wearable ring device.

16. The method of claim 1, wherein the wearable device collects the physiological data from the user based on arterial blood flow.

17. An apparatus for automatically detecting restorative moments, comprising:
a processor;
memory coupled with the processor; and
instructions stored in the memory and executable by the processor to cause the apparatus to:
receive physiological data associated with a user from a wearable device, the physiological data comprising at least heart rate data and temperature data;
identify that the heart rate data is less than or equal to a heart rate threshold for at least a portion of a time interval;
identify that the temperature data is within a temperature range of a baseline temperature associated with the user for at least the portion of the time interval;
identify a restorative moment for the time interval that the user is in a relaxed state based at least in part on the heart rate data being less than or equal to the heart rate threshold and the temperature data being within the temperature range of the baseline temperature; and
cause a graphical user interface of a user device to display an indication of the restorative moment.

18. The apparatus of claim 17, wherein the instructions are further executable by the processor to cause the apparatus to:
identify the baseline temperature associated with the user based at least in part on receiving the physiological data, wherein the baseline temperature comprises a nighttime temperature baseline.

19. The apparatus of claim 17, wherein the physiological data further comprises movement data, and the instructions are further executable by the processor to cause the apparatus to:
identify that the movement data is within a range of a baseline movement associated with the user for at least the portion of the time interval, wherein identifying the restorative moment for the time interval is based at least in part on the movement data being within the range of the baseline movement.

20. The apparatus of claim 17, wherein the physiological data further comprises heart rate variability data, and the instructions are further executable by the processor to cause the apparatus to:
identify that the heart rate variability data is within a range of a baseline heart rate variability associated with the user for at least the portion of the time interval, wherein identifying the restorative moment for the time interval is based at least in part on the heart rate variability data being within the range of the baseline heart rate variability.
